(19) United States
(12) Patent Application Publication
(10) Pub. No.: US 2008/0200372 A1
(43) Pub. Date: Aug. 21, 2008

(54) Glycosaminoglycan Peptides Derived From Connective Tissues And Use Thereof In The Prevention Of Arthritis And Other Degenerative Medical Conditions

(75) Inventor: Peter Ghosh, New South Wales (AU)

Correspondence Address: NIXON & VANDERHYE, PC, 901 NORTH GLEBE ROAD, 11TH FLOOR, ARLINGTON, VA, 22203, US

(73) Assignee: Institute of Nutraceutical Research Pty Ltd., Brookvale, NSW (AU)

(21) Appl. No.: 10/586,255

(22) PCT Filed: Jan. 14, 2005
(86) PCT No.: PCT/AU05/00044

§ 371 Date: Sep. 11, 2006

(30) Foreign Application Priority Data
Jan. 16, 2004 (AU) 2004 900250

(51) Int. Cl. A61K 38/14; C07K 9/00; A61P 19/02; A61P 19/04; A61P 37/02

(52) U.S. Cl. 514/8; 530/322

(57) ABSTRACT

The inventors sought to provide compositions and methods of prophylaxis to prevent these harmful immune responses in patients and to prevent the related undesirable symptoms biological tissue such as for example inflammation, cell injury, tissue injury, tissue degradation, tenderness, redness, joint stiffness, joint swelling, restricted mobility and reduced strength. The inventors found that prophylactic therapy of an animal comprising administering a pharmaceutical composition comprising a GAG-peptide complex, optionally together with a connective tissue derived polypeptide, prevented the onset of at least one undesirable symptom.

### CROSS-REFERENCE TO RELATED APPLICATIONS

The present application claims priority from Provisional Patent Application No 2004900250 filed on 16 Jan. 2004, the content of which is incorporated herein by reference.

### TECHNICAL FIELD

The present invention relates to methods for the prevention of inflammatory conditions of connective tissues such as arthritis, dermatitis, tendonitis, vascularitis and discitis.

### BACKGROUND ART

Inflammatory conditions of connective tissues such as: dermatitis, tendonitis, discitis rheumatoid arthritis (RA), osteoarthritis (OA), etc are a major cause of morbidity worldwide. These conditions have a substantial influence on health and quality of life and inflict an enormous burden on public health systems.

At sites of inflammation, connective tissues are infiltrated by monocytes, lymphocytes, polymorphonuclear leukocytes and other white cells. These cells are abundant sources of cytokines, prostanoids, procoagulant factors, proteinases and oxygen-derived free radicals (including nitric oxide radical [NO]). The pro-inflammatory mediators can directly and indirectly cause degradation of the connective tissues resulting in the loss of the their structural integrity and thus mechanical functions. This inflammatory cell mediated tissue breakdown is generally proceeded by increased vascular permeability resulting in oedema and the release of histamine and neuro-peptides which together with prostaglandins contributing to the clinical symptoms of pain and swelling.

In RA and OA it is the articular cartilage and bone of the synovial joints that are the connective tissues that are targeted by the mediators released from inflammatory cells and activated synovial cells. The cartilage extracellular matrix, particularly the collagens and PGs are the substrates for the proteinases released from these invading cells. However, extensive matrix breakdown also occurs due to the action of proteinases and NO free radicals released from the local chondrocytes by their interaction with cytokines, eg interleukin-1, originating from the inflamed synovium.

The molecular degradation products from the cartilage matrix, as well as cartilage detritus itself, are released into the synovial fluid enabling them to interact with the inflammatory cells of the cavity lining. Since these cartilage fragments originate from an immunologically privileged environment, they are seen by the local macrophage and inflammatory cells as ‘foreign’ (auto antigens) and can augment the immune response thereby perpetuating the synovial inflammation. In this context it should be noted that while RA has been recognised as an autoimmune disease for a number of years it is only recently that autoimmunity has been considered to play a pathogenic role in OA [Nishioka K, Autoimmune response in cartilage-derived peptides in a patient with osteoarthritis, Arthritis Research and Therapy, 6:6-7, 2004.

The agents most commonly used to treat RA, OA, and indeed many other inflammatory conditions, are the steroidal and non-steroidal anti-inflammatory drugs (NSAIDS). However, these drugs mainly relieve the symptoms arising from these diseases and have been shown to exhibit negligible beneficial effects in reducing the extent of breakdown of joint articular cartilage or subchondral bone. In fact there are reports that some NSAIDs may exacerbate the loss of cartilage and bone in arthritic joints by inhibiting cellular repair mechanisms and homeostasis. The chronic use of NSAIDs is also known to be associated with other deleterious side effects including a high risk of gastrointestinal bleeding. More recently it has been shown that NSAIDs with selective Cyclooxygenase-2 (COX-2) enzyme inhibitory activity (eg Vioxx® and Celebrex®), in which gastrointestinal bleeding was reduced, increased the risk of thrombosis and myocardial infarctions in RA and OA patients.

Broad acting immunosuppressants such as Cyclosporine A, Azathioprine, Cyclophosphamide, and Methotrexate, are used as second-line agents to treat chronic RA and other rheumatoid diseases, the rationale being to modulate the cellular aspects of the autoimmune reaction. Although the second-line agents may be effective in this regard, their low specificity and high potency are frequently associated with adverse severe side effects, including the development of neoplasias, destruction of bone marrow cells and liver and kidney toxicities. Furthermore, these drugs have the undesirable consequence of depressing the patient's immune system, which carries the risk of severe infectious complications and like the NSAIDs have no beneficial effects on cartilage.

Another therapeutic approach to the treatment or prevention of autoimmune disorders, such as RA and OA, includes suppression of the immune system in an auto-antigen specific manner (i.e. antigen-restricted tolerance). This concept has found limited application in the treatment of autoimmune diseases including rheumatoid patients where immuno-tolerization to type II collagen, the most abundant protein of cartilage, has been used. Refinements of this approach have used selected peptide sequences of the auto-antigen which contain the immuno-dominant epitopes [Myers, L. K., Seyer, J. M. Stuart, J. M., Terato, K. David, C. S. and Kang, A. H. T-cell epitopes of type II collagen that regulate murine collagen-induced arthritis. J. Immunol. 151: 500-505, 1993]. However, it is difficult to determine the correct amounts of the auto-antigen (or selected peptides) to administer to individual patients as type II collagen and some of its fragments are arthritogens and incorrect dosing may be accompanied by significant risks of allergic/hyposensitivity reactions and worsening of the disease. For these and other reasons, the use of type II collagen as an immuno-tolerance therapy for the treatment of RA or OA in humans has found limited clinical currency [Trentham D E, Oral tolerization as a treatment of rheumatoid arthritis. Rheumatic Disease Clin North Amer, 24:525-536, 1998].

In other studies it has been established that single GAG chains such as chondroitin sulfate (ChS) are useful for reducing the symptoms of OA (U.S. Pat. No. 5,364,845, Nov. 15, 1994; U.S. Pat. No. 6,136,795, Oct. 24, 2000; U.S. Pat. No. 6,162,787, Dec. 19, 2000, U.S. Pat. No. 6,271,213, Aug. 7, 2001, U.S. Pat. No. 6,432,929, Aug. 13, 2002), however their mechanism of action in this regard is largely unknown. Under normal digestion conditions broad specificity protease like papain and bromelain and aqueous solutions of sodium or potassium hydroxide degrade cartilage PGs down to single ChS chains and because of this property these substances have been employed to manufacture ChS and other GAGs from cartilage commercially.

Experimental studies designed to determine the possibility that ChS may act by preventing the onset of arthritis have proven to be negative. In the study of Omata et al. (2000), [Effects of chondroitin sulfate-C on articular cartilage destruction in murine collagen-induced arthritis. Arzneim Forsch./Drug Res. 50: 148-153:2000] the effects of pre-administration of chondroitin sulfate at oral doses of 100, 300 and 1000 mg/kg for 14 days prior to inducing type II collagen (CII) arthritis (CIA) in mice failed to prevent the incidence of arthritis. However, the severity of the disease, once it was established, as determined by the extent of hind paw oedema, synovitis and destruction of the articular cartilage, was significantly reduced but only at the highest dose of chondroitin sulfate used (1000 mg/kg). Significantly, ChS administration had no effect, at any dose, on the delayed-type hypersensitivity (DTH) reaction, a known marker of cellular immunity. On the basis of these studies the authors concluded that ChS had no effect on the pathogenesis of the experimental arthritis but may have reduced cartilage destruction by inhibition of neutrophil-derived elastase released into the joints during the type II collagen induced inflammatory reaction.

### SUMMARY OF INVENTION

In work leading up to the present invention, the inventors studied the effects of harmful immune responses to antigens in patients suffering from conditions such as rheumatoid arthritis, osteoarthritis, tendonitis, discitis and dermatitis. The inventors sought to provide compositions suitable for use its vivo and methods of prophylaxis to prevent these harmful immune responses in animals and to prevent the related undesirable symptoms manifested in a connective tissue (including tissue within and around or proximal to the connective tissue) such as for example inflammation, injury, degradation, tenderness, redness, joint stiffness, joint swelling, restricted mobility and reduced strength. The inventors found that prophylactic therapy of an animal comprising administering a pharmaceutical composition comprising a GAG-peptide complex, optionally together with a connective tissue derived polypeptide, prevented the onset of at least one undesirable symptom. Furthermore the inventors found that administration of a pharmaceutical composition comprising a GAG-peptide complex optionally in combination with a connective tissue derived polypeptide, to naive animals, tolerized the animals to the antigenic components of cartilage and prevented the appearance of symptoms of arthritis and inflammation.

Compositions comprising a GAG-peptide complex have also been found to exhibit reduced anticoagulant activity relative to chondroitin sulfate preparations and are gastro-protective as evidenced by the subsequent administration of NSAIDs to the immunised arthritic animals where stomach lesions were diminished relative to a non-treated arthritis control group. Advantageously, compositions comprising a GAG-peptide complex therefore not only preferably prevent the onset of arthritis, inflammatory diseases and inflammation and symptoms thereof but preferably provide additional protection against gastric irritation and haemorrhage which might occur, for example, should a patient elect to supplement their treatment with ChS, steroidal or NSAIDs including low dose Aspirin which may be taken daily to prevent thrombosis and cardiovascular disease.

In one embodiment of the invention, at least one of the GAG-peptide complexes for use in the present invention comprise 2 or 3 GAG chains. The invention includes mixtures of GAG-peptides having various GAGs and numbers of GAGs attached to a peptide, wherein at least one of the GAG-peptide complexes comprises two or three GAGs attached to a peptide. In a preferred embodiment, two GAGs are attached to the peptide. In an alternate embodiment three GAG chains are attached to the peptide. The mixture may also include a single GAG chain attached to a peptide or a single GAG chain such as chondroitin sulfate. Alternatively or addition to single GAG chains and single chain GAG-peptides the mixture may further comprise GAG peptide complexes comprising more than three GAG chains.

Accordingly, in one aspect the present invention provides a pharmaceutical composition for preventing the onset of a symptom of a harmful immune response in an connective tissue of an animal to an antigen, said composition comprising one or more GAG-peptide complex in combination with a pharmaceutically acceptable carrier, wherein at least one GAG-peptide complex comprises 2 or 3 GAG chains. Preferably the pharmaceutical composition prevents the onset of one or more symptoms of arthritis or tissue inflammation such as for example cell injury, tissue injury, tissue degradation, redness, tenderness, swelling of joints, joint stiffness, reduced mobility and decreased strength.

More preferably the pharmaceutical composition prevents the harmful immune response such that in a preferred embodiment, the present invention provides a pharmaceutical composition for prophylaxis of an animal to prevent a harmful immune response to an antigen, said composition comprising one or more GAG-peptide complex in combination with a pharmaceutically acceptable carrier, wherein at least one GAG-peptide complex comprises 2 or 3 GAG chains. In one embodiment the antigen is an antigenic component of cartilage.

In another preferred embodiment, the pharmaceutical of the present invention induces tolerance in an animal to an antigenic component of cartilage, said composition comprising one or more GAG-peptide complex in combination with a pharmaceutically acceptable carrier, wherein at least one GAG-peptide comprises 2 or 3 GAG chains.

In addition, in a preferred embodiment the composition preferably comprises at least one connective tissue derived polypeptide.

According to the present invention the GAG-peptide complex and polypeptide for use in the invention are obtainable from connective tissue by subjecting the connective tissue to autolysis or limited hydrolysis under conditions and for a time, such that a mixture of GAG-peptide complexes and polypeptides (referred to herein as “GAG-peptide complex polypeptide mixture”) are released into an autolysis or hydrolysis medium and are recovered from the autolysis or hydrolysis medium.

Methods for the obtaining a GAG-peptide complex and/or polypeptide for use in the present invention are described, for example in PCT/AU03/00061 (in the name of the Applicant) and Australian Provisional Application no. 2003903037, which are incorporated herein by reference. PCT/AU03/00061 discloses a method for the production of GAG-peptides which are substantially free of DNA. Advantageously, the method described in PCT/AU03/00061 comprises use of endogenous enzymes, and does not require subjecting the cartilage product to proteolytic digestion by the addition of exogenous enzymes such as papain to degrade the protein core. Australian Provisional Application no. 2003903037 provides methods for preparing and recovering connective tissue derived polypeptides.

In one embodiment, the method for recovering the GAG-peptide complex and polypeptide mixture comprises

(i) incubating a connective tissue in an autolysis medium that provides a buffered pH range of between about pH 2.5 and about pH 8.5 for a time and under conditions sufficient to release at least one GAG-peptide complex and at least one polypeptide; and
(ii) recovering a mixture comprising at least one GAG-peptide complex and at least one polypeptide from the autolysis medium.

In another embodiment the method for recovering the GAG-peptide complex and polypeptide mixture comprises

(i) incubating a connective tissue in a hydrolysis medium for a time and under conditions sufficient to release at least one GAG-peptide complex and at least one polypeptide; and
(ii) recovering a mixture comprising at least one GAG-peptide complex and at least one polypeptide from the hydrolysis medium.

In one embodiment the hydrolysis medium is an alkaline hydrolysis medium, preferably aqueous. Preferably the alkaline hydrolysis medium comprises a concentration of alkali of about 0.1-2.0% w/v. In a preferred embodiment the concentration of alkali is 0.1%, 0.2%, 0.4%, 0.8%, 1.0%, 1.5% or 2%.

Preferably the method for recovering the GAG-peptide complex polypeptide mixture comprises neutralization of the mixture.

According to another embodiment, the GAG-peptide complex polypeptide mixture is subjected to fractionation to select at least one GAG-peptide complex and a fraction comprising at least one GAG-peptide complex of a preferred molecular weight of greater than 1000 Da.

Accordingly, in another embodiment the method for recovering the mixture of GAG-peptide complexes and polypeptides comprises

(i) incubating a connective tissue in an autolysis medium that provides a buffered pH range of between about pH 2.5 and about pH 8.5 for a time and under conditions sufficient to release a mixture of at least one GAG-peptide complex and at least one polypeptide;
(ii) recovering a mixture comprising at least one GAG-peptide and at least one polypeptide from the autolysis medium and
(iii) fractioning the mixture to obtain a GAG-peptide and a polypeptide having a molecular weight of greater than about 1000 Da.

In another embodiment the method for recovering the GAG-peptide complex polypeptide mixture comprises

(i) incubating a connective tissue in an aqueous alkaline hydrolysis medium for a time and under conditions sufficient to release at least one GAG-peptide complex and at least one polypeptide;
(ii) recovering a mixture comprising at least one GAG-peptide complex and at least one polypeptide from the hydrolysis medium and
(iii) fractioning the mixture to obtain a GAG-peptide and a polypeptide having a molecular weight of greater than about 1000 Da.

In yet another embodiment the GAG-peptide complex and polypeptide mixture is recovered and one or more GAG-peptide complexes are separated from the polypeptides. Accordingly in one embodiment, the methods described herein further comprise separating the polypeptide from one or more GAG-peptide complex and recovering at least one GAG-peptide complex comprising 2 or 3 GAG chains, and optionally recovering the polypeptide.

It is to be understood that in addition to comprising a GAG-peptide complex, the composition of the invention can further comprise one or more polypeptides separated and recovered according to the methods of the invention or by alternate means. The present invention clearly extends a composition comprising a GAG-peptide complex alone or in combination with a polypeptide derived from any source. In a preferred embodiment the polypeptide is connective tissue derived. Preferably the connective tissue derived polypeptide is obtained by a method described herein.

In another aspect, the present invention provides a method of prophylactic therapy of an animal to prevent the onset of a harmful immune response or at least one symptom of a harmful immune response in a connective tissue comprising administering the animal with an effective amount of a composition comprising one or more GAG-peptide complexes wherein at least one GAG-peptide complex comprises 2 or 3 GAG chains. Preferably the method of prophylactic therapy of the animal prevents the harmful immune response.

In one embodiment, the invention provides a method for preventing an autoimmune response to at least one antigenic component of cartilage comprising administering the animal with an effective amount of a composition comprising one or more GAG-peptide complexes wherein at least one GAG-peptide complex comprises 2 or 3 GAG chains.

In a preferred embodiment, the invention provides a method of inducing tolerance in an animal to at least one antigenic component of cartilage comprising administering the animal with an effective amount of a composition comprising one or more GAG-peptide complexes wherein at least one GAG-peptide complex comprises 2 or 3 GAG chains.

In another aspect, the invention provides for a use of a composition comprising one or more GAG-peptide complex and optionally at least one polypeptide in the manufacture of a medicament for preventing the onset of a harmful immune response or at least one symptom of a harmful immune response in a connective tissue, wherein at least one GAG-peptide complex comprises 2 or 3 GAG chains.

In one preferred embodiment the invention provides a use of one or more GAG-peptide complex and optionally at least one connective tissue derived polypeptide in the manufacture of a medicament for inducing tolerance in an animal to at least one antigenic component of cartilage, wherein at least one GAG-peptide comprises 2 or 3 GAG chains. Preferably at least one GAG-peptide and at least one polypeptide are obtainable from a connective tissue by subjecting a connective tissue particle to autolysis in the presence of an autolysis medium or hydrolysis medium such that a mixture of GAG-peptides and polypeptides are released from the connective tissue particle into the autolysis or hydrolysis medium, and recovering the mixture of GAG-peptide complexes and polypeptide. Preferably, recovering the mixture comprises neutralization of the mixture.

In one embodiment the antigenic component of the cartilage is a component of the extracellular matrix. Types II, IV, VI, IX and XI collagens and the proteoglycans are the most abundant components, and there are host of non-collagenous components (known and unknown), which unquestionably, are important and are antigenic, such as for example cartilage oligomeric protein (COMP), cartilage matrix protein (CMP), fibronectin, fibromodulin, chondroadherin, PHELP etc.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

FIG. 1 provides a summary schema for the separation Peptacans such as CaP into its GAG-peptide (GAG-P) and polypeptides components using ion-exchange media.

FIG. 2 provides a photograph of a SDS-Polyacrylamide Gel (SDS-PAGE) of Calcium Peptacan (CaP), GAG-peptides and Polypeptides obtained from CaP by tangental flow ultrafiltration (TFF) using cartridges with various protein molecular weight (kDa) cut-offs as described in the text: Lane A=standard protein MW markers; Lane A=Total polypeptides in CaP [5 mg/mL(10 microL)] obtained after ion exchange treatment; Lane B=same as lane A but at 10 mg/mL; Lane C=GAG−P30 which is the retentate obtained from CaP using the 30 kDa cut-off membrane [10 mg/mL(10 microL)]; Lane D=Dialysate obtained from a 30 kDa cut-off experiment after it had been subjected to further ultrafiltration using a 10 kDa cut-off membrane [10 mg/mL(10 microL)]; Lane E=Dialysate obtained from 10 kDa cut-off experiment after it had been subjected to further ultrafiltration using 1 kDa cut-off membrane [10 mg/mL(10 microL)], Lane F=same as Lane C but at 5 mg/mL(10 microL)]; Lane G=CaP [10 mg/mL(5 microL)]; Lane H=CaP [10 mg/mL(10 microL)].

FIG. 3 provides Superdex-200 gel permeation chromatographic profiles of CaP (Panel A), GAG-P, prepared by from CaP by the ion exchange method (Panel B), GAG-P30, the retentate prepared from CaP using the 30 kDa TFF cut-off membrane (Panel C) and GAG-P10 the dialysate obtained from the 30 kDa TFF cut-off experiment but retained after further ultrafiltration of the dialysate using a 10 kDa cut-off TFF membrane (Panel D). The column was eluted with 0.25M NaCl at a flow rate of 11.0 mL/minute. Fractions (1.0 mL) were collected and assayed for the levels of sulfated glycosaminoglycans using the method of Farndale et al. 1986 [Farndale R W, Buttle D J and Barrett A J. Improved quantitation and discrimination of sulfated glycosaminoglycans by use of dimethylmethylene blue. Biochim. Biophys. Acta: 883, 173-177, 1986]. Note that ultrafiltration of CaP through the 30 kDa membrane removes the majority of ChS present.

FIG. 4 provides the a diagrammatic representation of the toleragenic and prophylactic protocols used to evaluate the anti-inflammatory and anti-arthritic activity of CaP and GAG-peptides described herein using the Collagen Induced Arthritis (CIA) and Adjuvant Induced Arthritis (AIA) rat models.

FIG. 5 provides Superdex-200 gel permeation chromatographic profiles of undegraded proteoglycans (PGs) extracted from bovine tracheal cartilage using 4M guanidium chloride (GuHCl) (Panel A), CaP (Panel B), the GAG-peptides produced by limited hydrolysis of bovine tracheal cartilage with bromelain (Panel C) and Chondroitin Sulfate (ChS) standard (Panel D). The column was eluted with 0.25M NaCl at a flow rate of 11.0 mL/minute. Fractions (1.0 mL) were collected and assayed for the levels of sulfated glycosaminoglycans using the method of Farndale et al, 1986 [Farndale R W, Buttle D J and Barrett A J. Improved quantitation and discrimination of sulfated glycosaminoglycans by use of dimethylmethylene blue. Biochim. Biophys. Acta: 883, 173-177, 1986]. Note that the GAG-peptides produced by limited hydrolysis with bromelain using the conditions described herein consist mainly of molecular species of sizes similar to CaP and ChS.

FIG. 6 provides a Superdex-200 gel permeation chromatographic profile of native proteoglycans (PGs) extracted from bovine tracheal cartilage using 4M Guanidium Chloride (GUHCl) (Panel A), the GAG-peptides produced by limited hydrolysis of bovine tracheal cartilage with 0.1% aqueous sodium hydroxide at 37° C., for 24 hours (Panel B), the GAG-peptides produced by limited hydrolysis of bovine tracheal cartilage with 0.1% aqueous sodium hydroxide at 37° C., for 26 hours (Panel C), the GAG-peptides produced by limited hydrolysis of bovine tracheal cartilage with 0.1% aqueous sodium hydroxide at 37° C., for 28 hours (Panel D), the GAG-peptides produced by limited hydrolysis of bovine tracheal cartilage with 0.1% aqueous sodium hydroxide at 37° C., for 30 hours (Panel E), and the GAG-peptides produced by limited hydrolysis of bovine tracheal cartilage with 0.1% aqueous sodium hydroxide at 37° C., for 44 hours (Panel F). The column was eluted with 0.25M NaCl at a flow rate of 11.0 mL/minute. Fractions (1.0 mL) were collected and assayed for the levels of sulfated glycosaminoglycans using the method of Farndale et al, 1986 [Farndale R W, Buttle D J and Barrett A J. Improved quantitation and discrimination of sulfated glycosaminoglycans by use of dimethylmethylene blue. Biochim. Biophys. Acta: 883, 173-177, 1986]. Note that the GAG-peptides produced by the alkaline limited hydrolysis with sodium hydroxide for 30 hours consist mainly of molecular species of sizes similar to CaP and ChS.

FIG. 7 provides a histogram showing the toleragenic effects of 7 days pre-administration of no drug (control), Chondroitin Sulfate (ChS) at 20 mg/kg, Calcium Peptacan (CaP) at 10 mg/kg or type II Collagen (Coll-II) at 10 mg/kg on arthritis development in the rat collagen induced arthritis (CIA) model as determined by rear paw swelling (mm), front paw inflammation and arthritis scores measured on day 15. Note that while both CaP and Coll-II were highly active in this animal model, ChS showed no activity.

FIG. 8 provides a histogram showing the prophylactic effects of 15 days administration of no drug (control), Glucosamine Sulfate (GmSO4), Glucosamine Hydrochloride (GmHCl) both at 200 mg/kg and Calcium Peptacan (CaP) at 20 mg/kg on arthritis development in the rat collagen induced arthritis (CIA) model as determined by rear paw swelling (mm), front paw inflammation and arthritis scores measured on day 15. Note that CaP was the only preparation that demonstrated anti-arthritic activity in this model.

FIG. 9 provides a histogram showing the toleragenic effects of 7 days pre-administration of no drug (control), Calcium Peptacan (CaP) at 20 mg/kg and Glycosaminoglycan Peptide (GAG-P), prepared from CaP by the ion exchange method, at 20 mg/kg on arthritis development in the rat collagen induced arthritis (CIA) model as determined by rear paw swelling (mm), front paw inflammation and arthritis scores measured on day 15. Note that at this period of assessment, CaP and GAG-P exhibit similar anti-arthritic potency.

FIG. 10 provides a histogram showing the toleragenic effects of 7 days pre-administration of no drug (control), Calcium Peptacan (CaP) at 20 mg/kg and Glycosaminoglycan Peptide (GAG-P), prepared from CaP by the ion exchange method, at 20 mg/kg on arthritis development in the rat collagen induced arthritis (CIA) model as determined by rear paw swelling (mm), front paw inflammation and arthritis scores measured on day 18. Note that at this period of assessment that the tolergenic activity of GAG-P was maintained but that of CaP had diminished.

FIG. 11 provides a histogram showing the dose dependent 15 day prophylactic effects of CaP on arthritis development in the rat collagen induced arthritis (CIA) model as determined by rear paw swelling (mm), front paw inflammation and arthritis scores measured on day 15. Note that CaP only expressed significant prophylactic anti-arthritic activity at doses above 3.3 mg/kg. However, a reduction in front paw inflammation was only significant at doses of 20 and 200 mg/kg.

FIG. 12 provides a histogram showing the dose dependent 15 day prophylactic effects of CaP and the GAG-P prepared from CaP on arthritis development in the rat collagen induced arthritis (CIA) model as determined by rear paw swelling (mm), front paw inflammation and arthritis scores measured on day 15. Note that the GAG-P was equipotent to CaP at 20 mg/kg in this model.

FIG. 13 provides a histogram showing the dose dependent 15 day prophylactic effects of CaP and the GAG-P prepared from CaP on arthritis development in the rat collagen induced arthritis (CIA) model as determined by rear paw swelling (mm), front paw inflammation and arthritis scores measured on day 18. Note that GAG-P at 20 mg/kg and CaP at 200 mg/kg were the only two concentrations that were significantly active in reducing rear paw joint swelling at this time period.

FIG. 14 provides a histogram showing the 15 day prophylactic effects of 200 mg/kg doses of GAG-P, GAG-P10, the dialysate obtained from the 30 kDa TFF cut-off experiment but retained after further ultrafiltration of the dialysate using a 10 kDa cut-off TFF membrane, and GAG-PLH, prepared by limited aqueous sodium hydroxide hydrolysis of bovine tracheal cartilage, on arthritis development in the rat collagen induced arthritis (CIA) model as determined by rear paw swelling (mm), front paw inflammation and arthritis scores measured on day 15. Note that GAG-P10 demonstrated lower prophylactic anti-arthritic activity than GAG-P and GAG-PLH when used at the doses of 200 mg/kg.

FIG. 15 provides a histogram showing the 15 day prophylactic effects of CaP (at 20 and 200 mg/kg) and GAG-P (20 mg/kg), prepared from CaP, on arthritis development in the rat adjuvant arthritis model (AIA), as determined by rear paw swelling (mm), front paw inflammation and arthritis scores measured on day 15. Note that GAG-P at 20 mg/kg was as active as CaP at doses of 200 mg/kg in this model.

FIG. 16 provides a histogram showing the 15 day prophylactic effects of CaP (at 20 and 200 mg/kg) and the GAG-P (20 mg/kg), prepared from CaP, on arthritis development in the rat adjuvant arthritis model (AIA) as determined by rear paw swelling (mm), front paw inflammation and arthritis scores measured on day 18. Note that, apart from front joint inflammation, CaP at doses of 200 mg/kg maintained a longer anti-arthritic effect than GAG-P (at 20 mg/kg) in this model.

FIG. 17 shows graphically the relative anticoagulant activities of CaP and the glycosaminoglycan peptide (GAG-P), isolated from CaP using the ion exchange method described in the text relative to 2 standard preparations of Chondroitin Sulfate (ChS) obtained from Bioiberica (ChS #1) and Sigma Chemical Co (ChS #2) respectively. In vitro anticoagulant activities were determined from their activated partial thromboplastin times (aPTT) (seconds) determined using citrated human plasma as described in the text. The strongest anticoagulant effect was observed with the 2 ChS preparations. CaP and GAG-P exhibited less than 1/loth the anticoagulant potency of the chondroitin sulfates on a weight basis in this assay.

FIG. 18 provides a table (Table 1) of the results for testing gastroprotective activity of Calcium Peptacan in the Rat CIA Arthritis Model against haemorrhagic stomach lesions produced by oral administration of the non-steroidal anti-inflammatory drug, Ibufrofen (50 mg/kg) on day 20.

FIG. 19 provides a table (Table 2) of the results for testing the topical anti-inflammatory activity of GAG-peptide preparations in a cream base. The GAG-peptide was prepared by limited hydrolysis of cartilage. The GAG-peptide composition showed higher anti-inflammatory activity than a preparation with no active ingredient.

### DETAILED DESCRIPTION OF THE INVENTION

### Prophylaxis to Prevent a Harmful Immune Response

As referred to herein prophylaxis is the prevention of disease, or the preventive treatment of a recurrent disorder. Preferably a prophylactic therapy is a measure designed to prevent disease and maintain health, in the form of a pharmaceutical composition or medicament or method of preventative treatment or therapy.

Immune responses are well known to include activation and involvement of various cell types such as for example antigen-presenting cells (APCs) such as macrophages and dendritic cells; the activation and proliferation of antigen-specific B-lymphocytes; the activation and proliferation of antigen-specific T-lymphocytes; and the production of antibody molecules, cytotoxic T-lymphocytes (CTLs), activated macrophages and NK cells, and cytokines.

As used herein a harmful immune response refers to an immune response which directly or indirectly causes or results in undesirable damage or injury to cells and/or tissue. For example in some circumstances the migration of inflammatory cells including polymorphonuclear leukocytes, macrophage and lymphocytes into and around the connective tissue can cause unwanted damage by the release of destructive enzymes, free radicals, chemokines and pro-inflammatory cytokines. These enzymes and endogenous factors can lead to the breakdown of the connective tissue extracellular matrix, resident cell death, angiogenesis and the establishment of chronic disease.

Harmful immune responses are observed in a number of degenerative diseases of connective tissue. The present invention provides methods and pharmaceutical compositions for the prevention of harmful immune responses which have an outcome of injury and lead to further damage as seen in diseases of the connective tissue such as for example arthritis, dermatitis, tendonitis, vascularitis and discitis.

Another example of a harmful immune response is one in which an antigen which is normally tolerated by an animal elicits an immune response in a hypersensitive animal (for example an allergen such as grass, dust or pollen). The immune system attacks the antigen vigorously, causing an inflammation which can be far more harmful than the antigen alone. Accordingly, in one example of the invention the pharmaceutical composition will prevent a harmful immune response in for example a hypersensitive animal.

In another example, a harmful immune response is activated in some cancers wherein a tumour cell activates the immune system and solicits an immune response to harness other cell types to release cellular factors such as enzymes. These cellular factors are thought to help the cancer cells to migrate and spread the cancer. Accordingly, preferably the compositions and methods of the invention prevent such harmful immune responses.

With regard to autoimmune disease, this refers to a disease comprising a harmful immune response directed at a self-antigen or epitope thereof. Autoimmune diseases are characterised by a humoral (eg., antibody-mediated), cellular (eg., cytotoxic T-lymphocyte-mediated), or a combination of both types of immune responses to epitopes on self-antigens. The immune system of the affected animal activates inflammatory cascades aimed at cells and tissues presenting those specific-self antigens. The destruction of the antigen, tissue, cell type or organ attacked gives rise to further symptoms of the disease. In one example of the present invention the autoimmune disease is for example any of rheumatoid arthritis, osteoarthritis, disc degeneration, and dermatitis.

The terms self-antigens or auto-antigens are used interchangeably to refer to an antigen that is endogenous to an animal's physiology, that is recognised by either the cellular component (eg T-cell or B-cell receptors) or humeral component (antibodies) of that animal's system.

Preferably, the pharmaceutical composition according to the present invention is one which when administered to a naive animal, prevents at least one symptom of a harmful immune response in connective tissue to an antigen, such as for example inflammation, cell injury, tissue injury and degradation, tenderness, redness, soreness, joint tenderness, joint swelling, joint stiffness, restricted mobility, or strength reduction.

As used herein to “prevent at least one symptom” refers to defending against or inhibiting a symptom, delaying the appearance of a symptom, reducing the severity of the development of a symptom, and/or reducing the number or type of symptoms suffered by an animal, as compared to not administering a pharmaceutical composition comprising a GAG-peptide complex. Accordingly, throughout this discussion, it will be understood that any clinically or statistically significant attenuation of even one symptom of a musculoskeletal degenerative condition pursuant to the treatment according to the present invention is within the scope of the invention.

By “naïve animal” is meant that preferably the animal does not present two or more symptoms of a musculoskeletal degenerative condition, more preferably the animal does not present a symptom of a musculoskeletal degenerative condition.

As used herein “degenerative disease”, “degenerative condition” or “degenerative disorder” are used interchangeably to refer to conditions that are characterised by a breakdown of a biological tissue, more particularly a connective tissue. Connective tissue refers to those animal tissue that supports organs, fills spaces between them, or performs mechanical functions such as connecting muscles to bone (tendons and ligaments) or providing low friction weighing surface as in articular cartilage. Connective tissues are characterized by their relatively avascular matrices and low cell densities. The most abundant connective tissues are the dermis, reticular stroma, muscle, cartilage and bone. The scope of the present invention clearly extends to tissue which is within, around, proximal or related to connective tissue.

The term “tissue” as used herein refers to matrices which contain similarly specialised cells that perform a common function. As used herein, tissue is intended to include an organ composed of a given tissue, and to the cells animally or collectively that compose the tissue.

### Tolerance

As used herein “tolerance” refers to the active state of specific immunologic nonresponsiveness induced by prior exposure to an antigen. Further, the terms “immunotherapy” and “tolerance therapy” refer to any general method resulting in tolerance or immunological prophylaxis. In vivo, these therapies typically entail a series of topical, parenteral or oral administrations of the immunogenic material over an extended period of time.

Experimentally induced tolerance may be defined as a state in which an animal will fail to respond to an antigen that will normally be immunogenic. Immunologic tolerance does not simply reflect the absence of an immune response, but rather an active response of the immune system that exhibits antigenic specificity and memory—the hallmarks of any immune response. In experimentally induced tolerance a foreign antigen is administered under certain conditions that promotes a state of tolerance rather than immune activation. Antigen structure, dosage and route of administration each partly determine whether the response of the immune system will lead to immunity or tolerance. Experimental evidence demonstrating the role of these factors is provided in J. Kuby in Immunology, 2nd ed, WH Freeman and Company, 1994, Chapter 16.

Oral tolerisation of an animal comprises orally administering an antigen to alter the response of the immune system. Oral tolerisation is an effective method of inducing peripheral T-cell tolerance such that mature lymphocytes in the peripheral lymphoid tissues are rendered non functional or hyporesponsive by the prior oral administration of an antigen. This therapeutic approach involves the participation of the gut-associated lymphoid tissue (GALT), a tissue comprising Peyer's patches, intraepithelial cells and villi containing epithelial cells which is a well organised immune network. All of the known mechanisms for tolerance induction, including clonal anergy, clonal deletion, and regulation by IL-4, IL-10 or TGF-beta-mediated active suppression appear to have a role in oral tolerance. It is thought that low doses of antigen induce the generation of active suppression, via regulatory T cells in the GALT. Conversely, higher doses of antigen appears to induce clonal anergy or clonal deletion. This phenomenon is of particular interest in T cell mediated autoimmune disease such as RA.

### Inflammatory Diseases of Connective Tissue Disease Involving a Harmful Immune Response

Examples of diseases of connective tissues which are thought to involve a harmful immune response include for example dermatitis, tendonitis, vasculitis OA and RA.

Dermatitis includes a wide range of inflammatory skin conditions including actinic dermatitis, contact dermatitis, rhus dermatitis, and rosacea. Tenosynovitis and vasculitis are terms used to describe inflammatory cell invasion and degenerative changes in the tendons sheath and blood vessel walls respectively, while tendinitis and discitis involve inflammation of the connective tissues themselves.

The aetiology of OA is considered to be multi-factorial with ageing, mechanical, hormonal and genetic factors all contributing to varying degrees. Osteoarthritis emerges as a clinical syndrome when these etiological determinants result in sufficient joint tissue damage to cause synovial inflammation and the appearance of the symptoms of pain and impairment of joint function. The joints of OA patients are characterized radiologically by joint space narrowing due to loss of articular cartilage and extensive bone re-modelling including the formation of osteophytes at the joint margins.

The rheumatoid diseases are a group of complex maladies in which arthritis is a common manifestation. They are all systemic diseases that have an underlying genetic, enviromental, endocrine and immunological aetiology but only in RA is chronic inflammation of the synovial lining and capsule of joints the primary and most relevant clinical finding. Inflammatory reactions within the dermis are common manifestations of psoriasis, scleroderma and systemic lupus erythematosus (SLE) but other sites such as the lung, heart and joints are also involved. The skin, eye and mucous membranes of the mouth and genitourinary tract become inflamed in Sjogren's Syndrome, Reiter's syndrome and Behcet's Syndrome but these syndromes are bettered classified as a group of multi-system inflammatory disorders.

The aggressive synovial inflammation that characterises RA results in chronic pain, loss of function, and can ultimately lead to destruction of the joint and permanent disability. As already indicated RA is a systemic disease arising from immunological aberrations probably triggered by genetic, endocrine and/or environmental factors. Moreover, the presence of T-cells subsets in the synovial fluid and membranes, antibodies to collagen type II in the serum, as well as other lines of evidence, strongly suggest that RA could be categorised as an autoimmune disease.

### Prophylaxis and Tolerance Therapy

### Models for a Disease State

GAG-peptide complex polypeptide mixtures and GAG-peptide complexes alone were prepared as described herein, and were evaluated for their anti-inflammatory, anti-arthritic and gastro protective activities in two well established rat arthritis models: (a) the type II collagen induced arthritis model (CIA) and (b) the adjuvant induced arthritis model (AIA).

Collagen induced arthritis is an experimentally induced autoimmune disease that can be elicited in susceptible strains of rodents and non-human primates by immunization with type II collagen (CII). Following immunization, the animals develop an auto-immune-mediated polyarthritis that shares several clinical, histological, and immunological features with the human autoimmune disease rheumatoid arthritis. As with rheumatoid arthritis, susceptibility to CIA in rodents is linked to the class II molecules of the major histocompatibility complex (MHC). The immune response to CII is characterized by both the stimulation of collagen-specific T cells and the production of high titers of antibody specific for both the immunogen (heterologous CII) and the auto antigen (mouse or rat CII). Histologically, mouse and rat CIA models are characterized by an intense synovitis that corresponds precisely with the clinical onset of arthritis. Within a few days of onset, erosion of cartilage and subchondral bone by pannus-like tissue is evident, and healing by fibrosis and ankylosis of involved joints follows slowly. Because of the important similarities between CIA and rheumatoid arthritis, this experimental model of autoimmune arthritis has been the subject of extensive investigation, see for example Creamer M, et al, Collagen-induced arthritis in rats, J Immunology, 149:1045-1053, 1992.

Adjuvant induced arthritis such as mycobacterium tuberculosis membrane (Mtb) induced arthritis is a model for rheumatoid arthritis having a chronic disease course influenced by both major histocompatibility complex and non-major histocompatibility complex genes. Studies have shown that the disease is T-cell dependent. Further information on this model is described for example in Whitehouse M W, Adjuvant induced polyarthritis in rats. In: Greenwald R A, Diamond H S, eds. Handbook of models for Rheumatic Diseases Vol 1. Boca Raton; CRC Press, 1988; 3-16.

According to the present invention the pharmaceutical composition of the present invention which comprises a GAG-peptide complex alone or in a mixture shows increased anti-inflammatory, antiarthritic and gastro protective activities in the CIA and AIA models when compared to glucosamine hydrochloride (Herron Pharmaceuticals, Brisbane, Australia), glucosamine sulfate (Blackmore Laboratories, Sydney, Australia), chondroitin sulfate (Bioiberica Ltd, Barcelona, Spain), type II collagen (Sigma Chemical Co, Sydney, Australia) as tested in the same models.

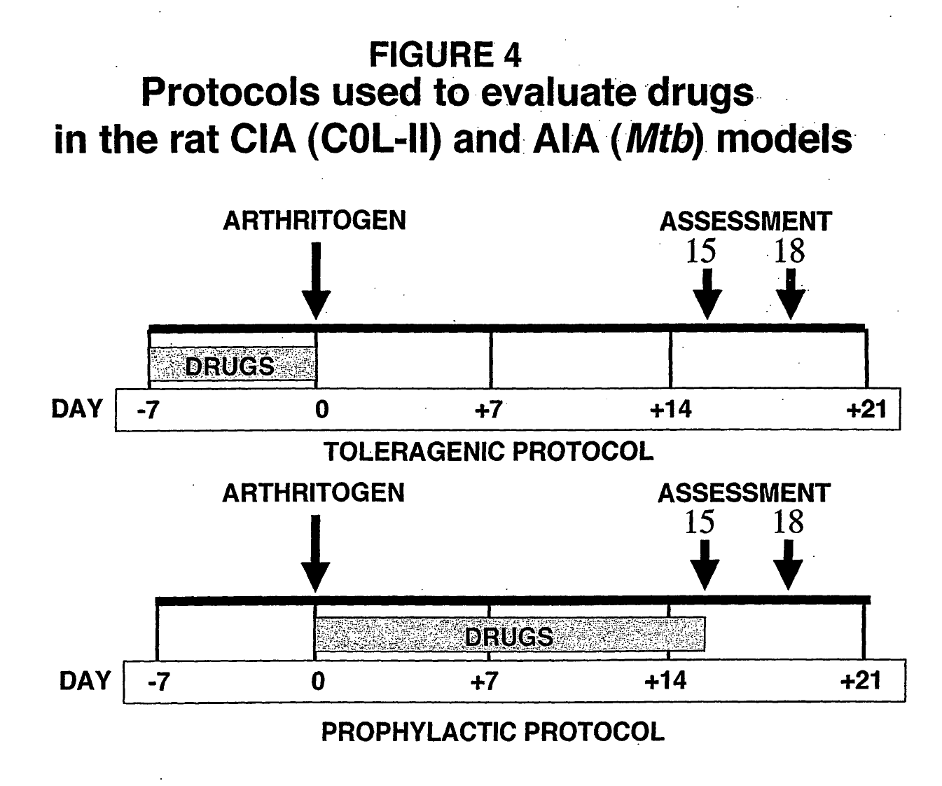

### Protocols to Evaluate Drugs in Animal Models (see FIG. 4)

According to the prophylactic protocol of evaluation, the pharmaceutical composition of the present invention is administered to the animals from the time the animals are injected with arthritogen and for a number of days thereafter. In one example the pharmaceutical composition is administered to the animal for between 1 and 15 days after administration of the arthritogen. In alternate examples of the invention the pharmaceutical composition is administered for example for 7, 10, 15 or 20 days after administration of the arthritogen. The scope of the present invention extends to the administration of the pharmaceutical composition for an appropriate period of time after administration of the arthritogen that is suitable to effect prophylaxis to prevent the onset of symptoms of a harmful immune response to the arthritogen.

In another example of the present invention, the tolerogenic protocol of evaluation of the pharmaceutical of the invention comprises administering the pharmaceutical composition to the animals prior to inducing arthritis by injection of the arthritogen. The pharmaceutical composition is administered for a period of time suitable to tolerize the animal. In one example the pharmaceutical composition is administered for 1 to 21 days prior to administration of the arthritogen. In one preferred example the pharmaceutical composition is administered for 1, 7, 10, 15 or 20 days prior to administration of the arthritogen. According to the toleragenic protocol for studying the activity of the pharmaceutical of the present invention, it is preferred that administration of the pharmaceutical composition ceases at the time that the arthritogen is administered.

### Pharmacological Activity of the Compositions of the Invention

Prophylactic Protocol in Rats with Collagen Induced Arthritis

In one embodiment of the present invention, a pharmaceutical composition comprising a GAG-peptide complex alone or a GAG-peptide complex polypeptide mixture prevents at least one symptom of rear paw inflammation, tail inflammation and fore paw inflammation in rats with collagen induced arthritis (CIA). Preferably administration of a pharmaceutical composition of the present invention comprising a GAG-peptide complex polypeptide mixture is more effective to prevent paw swelling and to prevent arthritis in animals when compared to no drug being administered, or Glucosamine Sulfate (GmSO4), or Glucosamine Hydrochloride (GMHCl) being administered. Preferably this is even the case where Glucosamine Sulfate (GmSO4) and Glucosamine Hydrochloride (GmHCl) are administered at 200 mg/kg and the pharmaceutical composition is administered at 20 mg/kg. In one example, the pharmaceutical composition of the present invention provides effective prophylaxis in rats with CIA at 10, 20 and 200 mg/kg. Accordingly, preferably the pharmaceutical composition is prophylactically effective when administered in an amount of about 10 mg/kg or more.

In one example a pharmaceutical comprising a GAG-peptide complex alone is equal to or more effective to prevent paw swelling than a composition which comprises a GAG-peptide complex polypeptide mixture. In one embodiment the pharmaceutical composition comprising a GAG-peptide complex alone provides a longer lasting prophylactic effect than a GAG-peptide polypeptide mixture. Preferably a composition comprising a GAG-peptide complex which is obtained by a method comprising autolysis is equally as effective as a GAG-peptide complex which is obtained by a limited hydrolysis method.

In one example of obtaining a GAG-peptide complex comprising 2 or 3 GAG chains, the method comprises fractioning a composition comprising at least one GAG-peptide complexes either alone or within a GAG-peptide polypeptide mixture to obtain a fraction comprising GAG-peptide complexes and polypeptides (if present) having a molecular weight of greater than 10,000 Da. Preferably, a fraction having a molecular weight of greater than 10,000 Da shows greater effective activity than a composition which comprises a fraction of GAG-peptide complexes of less than 10,000 Da.

In another example of the invention, the pharmaceutical composition preferably provides gastro protective effects against administration of a non-steroidal antiinflammatory drug (NSAID) such as for example ibuprofen. Preferably administration of the pharmaceutical composition prevents gastric irritation and preferably gastric bleeding in an animal who is administered a NSAID. Preferably the pharmaceutical composition provides gastro protective effects when administered 20, or even 4 days prior to administration of a NSAID.

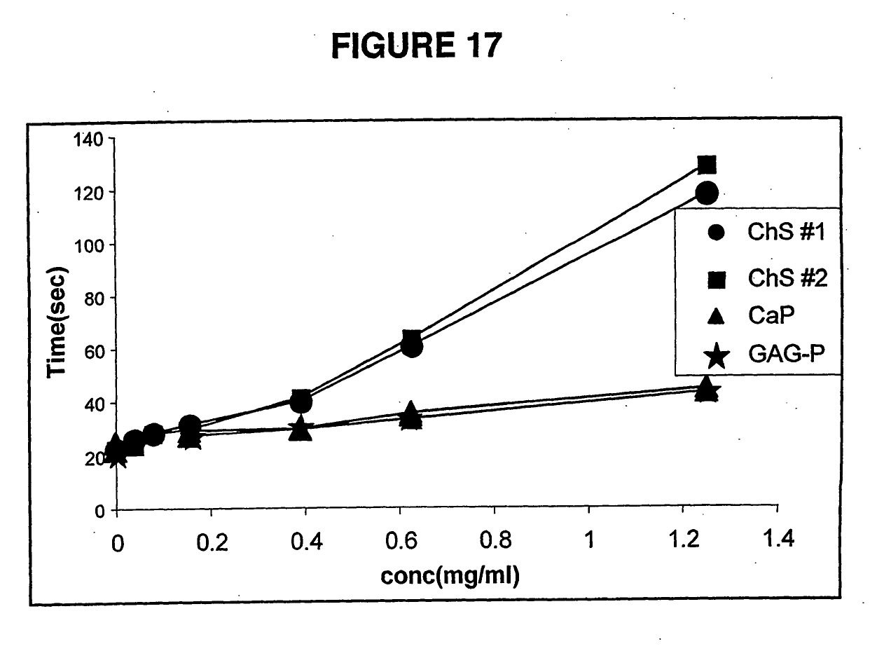

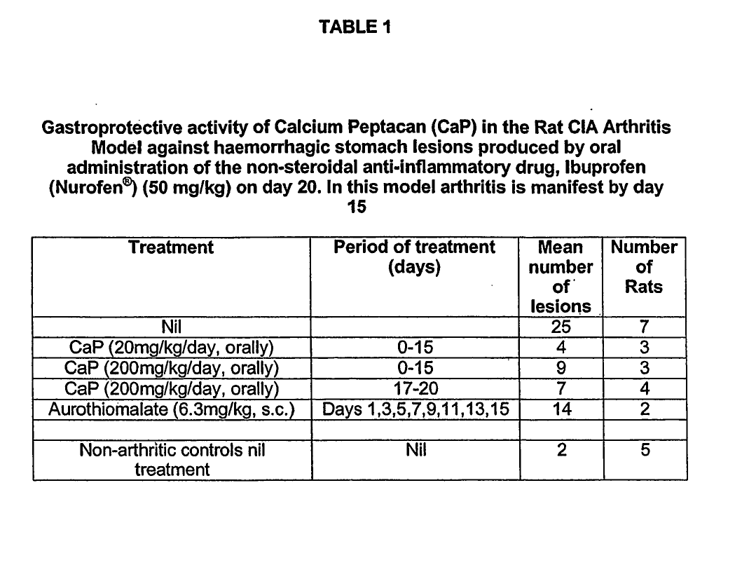

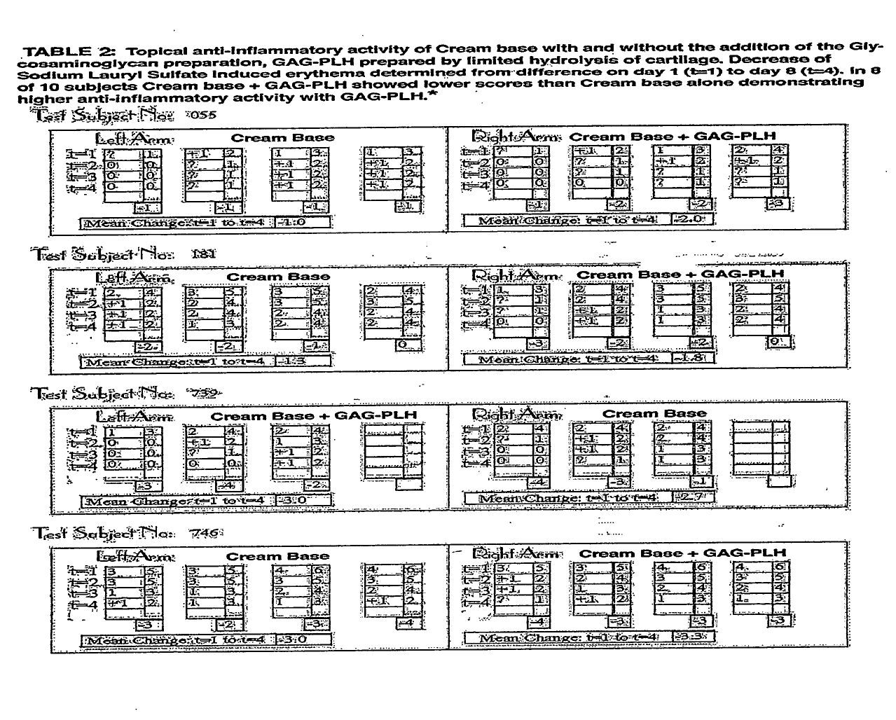

In another example the pharmaceutical of the invention comprising GAG-peptide complex alone or GAG peptide complex polypeptide mixture is less effective to promote bleeding compared to chondroitin sulfate when tested in an anti-coagulant assay, see FIG. 17.

Prophylactic Protocol in Rats with Adjuvant Induced Arthritis

In other embodiment, a pharmaceutical composition of the invention comprising a GAG-peptide complex alone or a GAG-peptide complex polypeptide mixture prevents at least one of the symptoms of rear paw inflammation, tail inflammation and fore paw inflammation in rats with adjuvant induced arthritis (AIA). Preferably the pharmaceutical composition prevents arthritis in the animal. In one example pharmaceutical compositions comprising a GAG-peptide complex alone or a GAG-peptide complex polypeptide mixture are equally as effective to prevent paw swelling and arthritis in rats with AIA. In another example where a greater period of time has elapsed since the arthritogen is administered, a composition comprising a GAG-peptide complex alone is more effective to prevent swelling (front paw) than a composition comprising a GAG-peptide complex polypeptide mixture. In another example a pharmaceutical comprising a GAG-peptide complex polypeptide mixture, is more effective to prevent swelling (rear paw) and overall arthritis symptoms than a composition which comprises a GAG-peptide complex alone.

Toleragenic Protocol in Rats with Collagen Induced Arthritis

In a preferred embodiment of the present invention, a pharmaceutical composition comprising a GAG-peptide complex alone or a GAG-peptide polypeptide mixture induces tolerance to the antigenic components of cartilage in rats with collagen induced arthritis (CIA). Preferably the tolerization of the rats to the onset of CIA with the pharmaceutical of the present invention is equally effective at inducing tolerance compared to the tolerizing effects of type II collagen when administered at the same dose. In one example, the pharmaceutical of the present invention provides greater tolerization to an althritogen than compared to chondroitin sulfate, glucosamine sulfate, glucosamine hydrochloride or to no tolerizing therapy.

Preferably, administration of the pharmaceutical according to the present invention is not arthritogenic when used to rechallenge the tolerized rats.

In another example a pharmaceutical composition comprising a GAG-peptide alone is effective to induce tolerance in a rat CIA model and shows a longer lasting effect than a GAG-peptide complex polypeptide mixture when administered at the same dose. In one example, the pharmaceutical composition of the present invention is effective to induce tolerance when administered in an amount of 10, 20 and 200 mg/kg. Accordingly, preferably the pharmaceutical composition is administered in an amount of about 10 mg/kg or more.

### Protection Against Arthritis, Inflammatory Diseases and Inflammation in Other Animals Including Humans

The effects observed for the GAG-peptide complex alone and GAG-peptide complex polypeptide mixture on inflammation and collagen induced arthritis and adjuvant induced arthritis in rats provide application for the GAG-peptide complex alone and GAG-peptide complex polypeptide mixture in the prevention of inflammation, inflammatory disease (particularly in inflammatory connective tissue diseases) and other diseases involving harmful immune responses such as RA, OA and dermatitis.

Rodent models of connective tissue disease are well known to the person skilled in the art of have being a good predictor for animal (including human) therapy. For example, rat models for collagen induced arthritis and pristane induced arthritis rats are described in Lu et al (1999) (supra). Murine collagen induced models of arthritis are also described in Myers et al (2002) (supra) and Omata et al (2000) (supra).

GAG-peptide complexes alone and GAG-peptide complex polypeptide mixtures which are useful in the present invention and that have been shown to prevent the appearance of symptoms such as inflammation in rats and/or tolerization of rats can be further tested for safety and efficacy in other animal models and then proceed to clinical trials in humans, if desired. Naturally, for veterinary applications, no clinical trial in humans is required. Those GAG-peptides and polypeptides that are safe and efficacious in animals or humans can be administered to an appropriate subject to tolerize the animal against the antigenic components of cartilage and preferably protect the animal against connective tissue inflammation and arthritis.

Pharmaceuticals and methods of the present invention are also suitable for use in conjunction with alternate treatments or for dermatological inflammatory and rheumatic diseases. As discussed herein above, one beneficial effect of the present composition is the decreased anticoagulant activity and gastro-protective effects and accordingly, in one embodiment the pharmaceuticals and methods of the present invention will benefit, for example, a patient who elects to supplement their arthritic disease treatment with chondroitin sulfate, steroids or NSAIDs.

In another example of the invention the pharmaceutical composition is also useful for preventing inflammation of the skin when applied topically. In one example the invention preferably provides a topically administered pharmaceutical composition which prevents inflammation induced by environmental factors such as actinic dermatitis (sunburn), contact dermatitis (allergy) or a from a combination of genetic, environmental and infectious organisms as in Rosacea. In another example preferably the pharmaceutical composition when administered topically prevents an autoimmune disease with dermatological manifestations. In yet another example the topical administration of the pharmaceutical composition of the invention prevents the progression and occurrence of skin cancers. In another example the pharmaceutical composition is capable of modulating a Th1-dominant T-cell cell mediated immunity. In a preferred embodiment a GAG-peptide complex which is useful in a pharmaceutical composition to be administered topically is obtained from a connective tissue polypeptide by a method comprising limited hydrolysis of a connective tissue.

GAG-Peptide Complexes Derived from Connective Tissue

Aggrecans of cartilage are composed of 20-50 proteoglycan (PG) subunits that are non-covalently bound to a hyaluronic acid (HA) filament. The magnitude of the binding of the PG subunits to the HA chain is augmented by the presence of link proteins located at globular end of the PG subunit. The PG subunit is composed of a protein core to which up to 100 glycosaminoglycans (GAG) chains are covalently attached. The most abundant GAGs that are attached to the protein core of the PG subunits of cartilage are the chondroitin-4- and chondroitin-6-sulfates (ChS) which are mostly located distal to the HA binding region of the core protein. Another GAG, keratan sulfate (KS) is mainly clustered along the region of the PG core protein closer to the region which interacts with HA and link proteins.

As described herein GAG-peptide complexes are obtainable by methods which comprise autolysis or limited hydrolysis to release a GAG-peptide polypeptide mixture in to a medium and recovering the GAG-peptide complex polypeptide mixture or the GAG-peptide alone. In one example non-covalently bound polypeptides are removed for example by the ion exchange method. In another example the GAG-peptide complex polypeptide mixture or the GAG-peptide complex alone is treated to select molecules according to their molecular size using ultra-filtration membranes and TFF.

The average molecular mass of a GAG-peptide complex can be assessed, for example, by using a Sephadex-200 high resolution gel exclusion column and compared to a known standard. According to the methods of the present invention, on average, the GAG-peptides have an approximate molecular weight of 32,000 Da.

The degree of sulfation, length and number of any GAG chain covalently attached to a peptide fragment may vary. In one example the invention comprises a mixture of GAG-peptides comprising different types and numbers of GAG chains. According to the present invention the composition comprises a GAG-peptide having two or three GAG chains attached to the peptide. The invention clearly extends to a composition comprising a mixture of GAG-peptide complexes having different numbers of GAG chains attached to a peptide including single chains, 2 chains, 3 chains or more. Preferably, the GAG is selected from chondroitin sulfate (ChS) or keratan sulfate.

As used herein the term “peptide” refers to a polymer of amino acid residues. The term also applies to amino acid polymers in which one or more amino acids are chemical analogues or modified derivatives of a corresponding naturally occurring amino acid. A GAG-peptide useful in the present invention preferably comprises a peptide of about 2-20 amino acids. Preferably, the peptide comprises about 5-15 amino acids, more preferably 10 amino acids. Alternatively, the peptide comprises 20-100 amino acids.

In one example, a GAG-peptide comprises 2 or 3 GAG chains attached to a peptide stub of about 10 amino acids. In a preferred example at least one GAG chain is chondroitin sulphate.

Methods of Obtaining GAG-Peptide Complexes and Polypeptides from Connective Tissue Autolysis

In one embodiment of the invention, GAG-peptide complexes and or polypeptides are derived from connective tissue by a process of autolysis. Accordingly, the invention includes a method for recovering a GAG-peptide complex and/or a polypeptide from connective tissue wherein connective tissue particles are subjected to autolysis by incubation in an autolysis medium such that a mixture of GAG-peptide complexes and polypeptides are released from the connective tissue particles into the autolysis medium. According to one embodiment of the invention, GAG-peptide complexes are recovered from the autolysis medium and separated from the polypeptides.

As used herein the term “autolysis” refers to the digestion of cellular components by endogenous hydrolases and proteinases released from lysosomes or associated with the cell and its pericellular matrix following cell death, causing self digestion of the tissue. A person skilled in the art will appreciate that the rate of autolysis will vary with many factors including pH, temperature, concentration, tissue type, tissue particle size and time of incubation.

Connective tissues suitable for use in the present invention includes for example: connective tissue of the cartilage, lung, skin, bone, ligament or tendon. In a preferred embodiment the connective tissue is of cartilage. Preferably the cartilage is tracheal, articular, auricular, nasal, sternal, rib skeletal, or antler cartilage. Cartilage may be however any type of cartilage or a mixture thereof.

Connective tissue may be obtained from any animal species having connective tissue such as for example human, bovine, ovine, porcine, equine, avian, cervine and piscine species. Preferably the connective tissue is bovine, ovine, porcine, cervine, shark or equine.

The connective tissue is treated and washed as required by methods known in the art to remove any adhering soft tissues. Preferably the connective tissue is reduced to a particle size. The connective tissue can be reduced to a particle size by means including, but not limited to, mincing, dicing, grinding and the like. In one example particle diameter is less than about 5 mm, preferably less than about 4 mm, more preferably less than about 3 mm. Most preferably, the particle diameter is about 0.1 mm to about 3 mm. In an alternate example the connective tissue is not reduced to a particle size.

The terms “incubation”, “incubate” or “incubating” mean to contact, suspend or maintain (a chemical or biochemical system) under specific conditions in order to promote a particular reaction.

The term “buffer” refers to a compound, usually a salt, which, when dissolved in an aqueous medium, serves to maintain the free hydrogen ion concentration of the solution within a certain pH range, when hydrogen ions are added or removed from the solution. A salt or salt solution is said to have a “buffering capacity” or to buffer the solution over such a range, when it provides this function. Generally a buffer will have adequate buffering capacity over a range that is within about ±1 pH unit of its pK. The salt is preferably a monovalent or divalent salt. Preferably the monovalent salt is selected from any one or more of hydrogen, sodium, potassium, or ammonium. Preferably a divalent salt selected from any one or more of calcium, magnesium, copper, or zinc. Most preferably the salt is calcium or magnesium.

Preferably the pH is in the range of about pH 2.5 to about pH 8.5, preferably about pH 3.5 to about pH 8.0, more preferably about pH 4 to about pH 7 and most preferably about pH 4.5 to about pH 7.

The term “condition” refers to other factors which affect the rate, efficiency and amount of autolysis, such as, for example, temperature and time. In one example the temperature conditions for carrying out the step of autolysis is in the range of from about 20° C. to about 45° C., preferably about 25° C. to about 45° C., more preferably about 32° C. to about 45° C., more preferably about 32° C. to about 40° C. most preferably about 37° C.

In one example, the autolysis proceeds for about 44-48 hours. In another example the autolysis proceeds for about 36-44 hours. In another example the autolysis proceeds for about 32-36 hours. In another example the autolysis proceeds for about 28-32 hours. In another example the autolysis proceeds for about 24-28 hours. In one example autolysis proceeds for about 16-24. In another example autolysis takes about 1 to about 16 hours. Preferably autolysis proceeds for about 16-44 hours, preferably 16-28 hours and most preferably 16-24 hours.

In one example, cartilage particles of about 1-3 mm are subject to autolysis in an aqueous medium at a pH of about 4-5 and temperature of about 32-45° C. for about 16-24 hours.

GAG-peptides and polypeptides can be recovered from the autolysis medium by well known methods. For example, methods of recovery include filtration to remove residual tissue particles from the autolysis media and recovery of the mixture of GAG-peptide complexes and polypeptides from the supernatant. In addition to, or alternatively, the mixture comprising GAG-peptide complexes and polypeptides is preferably neutralized by addition of an alkaline solution containing a cation. In one example the neutralised mixture is thereafter freeze dried. In an alternate embodiment the neutralised mixture is kept as a liquid. Neutralization of the GAG-peptide complex alone or GAG-peptide polypeptide mixture is preferably effective to make the composition useful as a pharmaceutical and stabilise the composition.

In other examples of recovering a mixture of GAG-peptide complexes and polypeptides the medium or supernatant is treated by precipitation with excess quantities of acetone, or aliphatic alcohols, such as, for example, ethanol or methanol. In addition to or alternatively, the method of recovery also comprises formation of water insoluble complexes with quaternary ammonium salts such as cetyl pyridinium, chloride. In a further example the method comprises separation or selection of the GAG-peptide complex using size exclusion or ion-exchange or other forms of column chromatography or membrane filtration technology.

The present invention clearly extends to any combination of methods suitable to obtain a GAG-peptide complex and/or polypeptide for use in the present invention.

### Limited Hydrolysis

An alternate method for obtaining a GAG-peptide complex and polypeptide from connective tissue comprises limited hydrolysis of connective tissue, such as cartilage, using an acid, base or by the action of an exogenous proteinase. According to the present invention the extent of hydrolysis with alkalis, acids or proteolytic enzymes is controlled to obtain the desired GAG-peptide complex comprising 2 or 3 GAG-chains by terminating the hydrolysis reaction before it comes to completion. The rate (or extent) of hydrolysis of a protein or polymeric carbohydrate substrate is dependent on a number of factors including: the concentration of the substrate, its physical form, the concentration of the proteinase, acid or base, the temperature of the hydrolysis medium, the presence and pH of a buffer, and the time course of the reaction.

Preferred proteinases useful in methods of the invention include for example cysteine proteinases such as papain, bromelain, ficin. Alternatively alkaline solutions such as for example hydroxides eg, sodium or potassium hydroxide are useful in a method of limited hydrolysis. In a preferred embodiment the concentration of a the alkali is 0.1%-2.5% (w/v)

Preferably the temperature for the method of hydrolysis is maintained at between 20° C. and 60° C. More preferably, the temperature is maintained at between 35° C. and 60° C. In one preferred embodiment the temperature is maintained for example at any one more of 37° C., 40° C., 43° C., 47° C., 50° C., 54° C. or 57° C.

The pH of the conditions for limited hydrolysis is also adjusted to control the rate of hydrolysis and depends on the hydrolysis medium used. For example, if a proteases is used then it is suitable to use a buffer having a pH preferably between about pH 6 and pH 7.5. In one example the pH of the buffer is pH 6 or 7.

Alternatively, where an alkaline hydrolysis medium is used in the hydrolysis method the pH of the medium in which hydrolysis proceeds is greater than pH 7, and preferably greater than pH 8. In alternate examples of the invention the pH is any alkaline pH. In one example the hydrolysis medium comprises 2% NaOH (0.5 M NaOH) which has a pH that is above pH 13.

The time course of the reaction is another element for controlling the limited hydrolysis reaction. Increasing the time period of the reaction provides more time for digestion of the GAG-peptide complexes and accordingly increasing the time of the reaction is expected to increase the proportion of GAG-peptide complexes and polypeptides having a smaller molecular weight in the mixture. Preferably the conditions are such that the maximum proportion of GAG-peptide complexes comprising 2 or 3 GAG-chains are obtained. Preferably, the limited hydrolysis reaction proceeds for between about 10 hours and 48 hours, more preferably between 24 hours and 44 hours. In several examples of the invention the limited hydrolysis method proceeds for any of 24 hours, 26 hours, 28 hours, 30 hours and 44 hours. In a preferred example the limited hydrolysis reaction is allowed to proceed for 30 hours.

One preferred example of the conditions of limited hydrolysis comprises limited hydrolysis of tracheal cartilage using an exogenous protease bromelain maintained at 58° C. in a sodium bicarbonate buffer pH=6.0 for 24 hours. In another example the conditions comprise limited hydrolysis of bovine tracheal cartilage with 0.1% aqueous sodium hydroxide at 37° C., for 30 hours.

Following limited hydrolysis the GAG-peptide complex polypeptide mixture is preferably treated to neutralise, separate, precipitate recover and/or fraction the mixture, as desired.

Accordingly in another example, limited hydrolysis of tracheal cartilage comprises using alkaline of 0.1% aqueous sodium hydroxide maintained at 37° C. for 44 hours followed by neutralisation using conventional mineral acids or organic acids such as acetic acid, tartaric acid, glucuronic acid, lactobionic acid or ascorbic acid.

Method of precipitation are within the scope of the invention and in one example the GAG-peptides obtained by the process of limited hydrolysis are precipitated and fractionated from the aqueous reaction solutions by stepwise addition of increasing concentrations of an aliphatic alcohol such as ethanol, or completely precipitated using a complexing agent, for example cetyl pyridinium chloride (CPC). The water insoluble CPC-GAG peptide complex so collected can then be treated for example with sodium thiocyanate to release the GAG-peptide back into solution.

Preferably, the desired GAG-peptide complex is separated from inorganic ions and fractionated according to size or charge using gel permeation chromatography, ion exchange chromatography or membrane filtration technologies.

### Fractionation

As used herein the term “fractionation” refers to the separation of a mixture in successive stages, each stage removing some portion of the one of the components of the mixture.

In one example the mixture of GAG-peptide complexes and polypeptides prepared by the methods described herein are subjected to fractionation by ultrafiltration using for example synthetic membranes or tangential flow ultrafiltration (TFF) cartridges with different molecular weight cut-offs. The GAG-peptide complexes prepared by these methods contain fractions of bioactive polypeptides originally present in the mixture, the nature of which is determined by the type of membrane or cartridge used for ultrafiltration. The GAG-peptide complexes comprising 2 GAG chains have an approximate molecular weight of about 32,000 Da.

Accordingly, in one example the mixture is fractionated to obtain at least one GAG-peptide complex and polypeptide with a molecular weight of greater than 30.000 Da. In one embodiment, the mixture is diafiltrated with a PLTK 30K regenerates cellulose cartridge.

In another example, the mixture is fractionated to obtain a GAG-peptide complex and polypeptide having a molecular weight of greater than 10,000 Da. In one embodiment the mixture is diafiltrated using a PTGC 10K polyether sulfone.

Alternatively, by subjecting the dialysate from the 30,000 Da ultrafiltration through a 10,000 Da membrane GAG-peptides and polypeptides with molecular weights between 30,000 Da and 10,000 Da are obtained. In other examples, the mixture is fractionated to obtain GAG-peptides and polypeptides of greater than or less than 20 kDa, 15 kDa, 5 kDa, or 1 kDa.

Preferably, the compositions and methods of the present invention comprise a mixture of GAG-peptide complexes and polypeptides having a molecular weight of greater than 10,000 Da. More preferably the mixture comprises GAG-peptide complexes and polypeptides having a molecular weight of greater than about 20,000 Da, more preferably greater than about 30,000 Da, and most preferably comprise at least one GAG peptide complex of about 32,000 Da.

### Separating and Recovering the GAG-Peptides and Polypeptides of the Invention

In addition to or in the alternative of fractionation, the polypeptides and GAG-peptide complexes are separated by methods such as for example ion-exchange, chromatography, or precipitation.

In one example, the mixture of a GAG-peptide complex and polypeptide is subjected to an ion exchange technique. In a preferred example the mixture is separated by treatment with ion exchange solid phase media, such as for example DEAE sepharose, or pre-swollen DEAE-Sepharose-6B.

In alternate methods of separation, the mixture is centrifuged and the supernatant recovered. In one example the supernatant is subjected to ultrafiltration, preferably using a 0.5 k Da cut-off membrane optionally in the presence of nitrogen gas to remove inorganic salts. In one example, the de-salted GAG-peptide complex solution is then freeze-dried and stored at −20° C.

It is understood that the embodiments of recovery, separation and fractionation apply mutatis mutandis to methods of autolysis and hydrolysis alike, and as appropriate.

### Preparation and Administration of Pharmaceutical Compositions

A pharmaceutical composition comprising a GAG-peptide alone or GAG-peptide complex polypeptide mixture that is suitable for administration (from whatever source derived) may be administered to a patient in need, by itself, or in pharmaceutical compositions where it is mixed with suitable carriers or excipient(s) at doses to treat or ameliorate a variety of disorders. Such a composition may optionally contain (in addition to GAG-peptide complex or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNFO, TNF1, TNF2, G-CSF, Meg-CSF, thrombopoietin, stem cell factor, and erythropoietin.

In further compositions, pharmaceutical compositions of the invention may be combined with other agents beneficial to the treatment of the disease or disorder in question. These agents include various growth factors such as epidermal growth factor (EGF), platelet-derived growth factor (PDGF), transforming growth factors (TGF-a and TGF-13), insulin-like growth factor (IGF), as well as cytokines described herein.

The pharmaceutical composition may further contain other agents which either enhance the activity of the pharmaceutical composition or other active ingredient or complement its activity or use in methods of the invention. Such additional factors and/or agents may be included in the pharmaceutical composition to produce a synergistic effect with the pharmaceutical composition or other active ingredient of the invention, or to minimize side effects. Conversely, a GAG-peptide or other active ingredient may be included in formulations of the particular clotting factor, cytokine, lymphokine, other hematopoietic factor, thrombolytic or anti-thrombotic factor, or anti-inflammatory agent to minimize side effects of the clotting factor, cytokine, lymphokine, other hematopoietic factor, thrombolytic or anti-thrombotic factor, or anti-inflammatory agent (such as IL-1Ra, IL-1 Hy1, IL-1 Hy2, anti-TNF, corticosteroids, immunosuppressive agents).

As an alternative to being included in a pharmaceutical composition of the invention including a GAG-peptide complex, a second pharmaceutical composition or a therapeutic agent may be concurrently administered with the first pharmaceutical composition (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in prevention of the relevant medical condition, or symptoms of the condition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of prophylaxis or use of the pharmaceutical composition of the present invention, a therapeutically effective amount of pharmaceutical composition or other active ingredient of the present invention is administered to a mammal to be treated, without necessarily having any condition. A pharmaceutical composition or other active ingredient of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, pharmaceutical composition or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering pharmaceutical composition or other active ingredient of the present invention in combination with cytokine(s), lymphokine(s), other hematopoietic factor(s), thrombolytic or anti-thrombotic factors.

### Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of pharmaceutical composition or other active ingredient used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Non-invasive administration to the patient is preferred.

The pharmaceutical compositions of the invention are administered by any route that delivers an effective dosage. The determination of a suitable route of administration and an effective dosage for a particular indication is within the level of skill in the art. Suitable dosage ranges for the pharmaceutical compositions of the invention can be extrapolated from these dosages or from similar studies in appropriate animal models. Dosages can then be adjusted as necessary by the clinician to provide maximal prophylactic benefit.

### Formulations

Pharmaceutical compositions for use in accordance with the present invention thus maybe formulated in a conventional manner using one or more physiologically acceptable carriers comprising excipients and auxiliaries which facilitate processing of the active compounds into preparations which can be used pharmaceutically. These pharmaceutical compositions maybe manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

When a therapeutically effective amount of the pharmaceutical composition or other active ingredient is administered orally, the pharmaceutical composition or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% pharmaceutical composition or other active ingredient of the present invention, and preferably from about 25 to 90% pharmaceutical composition or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of pharmaceutical composition or other active ingredient, and preferably from about 1 to 50% pharmaceutical composition or other active ingredient.

When a therapeutically effective amount of pharmaceutical composition or other active ingredient is administered by intravenous, cutaneous or subcutaneous injection, the pharmaceutical composition or other active ingredient will be in the form of a pyrogen free, parenterally acceptable aqueous solution. The preparation of such parenterally acceptable pharmaceutical composition or other active ingredient solutions, having due regard to pH, isotonicity, stability, and the like, is within the skill in the art. A preferred pharmaceutical composition for intravenous, cutaneous, or subcutaneous injection should contain, in addition to pharmaceutical composition or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the bather to be permeated are used in the formulation. Such penetrants are generally known in the art. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures.

Dyestuffs or pigments may be added to the tablets or dragee coatings for identification or to characterize different combinations of active compound doses. Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers maybe added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the composition and a suitable powder base such as lactose or starch.

The compositions maybe formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatony agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The amount of GAG-peptide complex or other active ingredient in the pharmaceutical composition of the present invention will depend upon the nature of the condition to be prevented, and on the nature of prior treatments which the patient has undergone. Ultimately, the attending physician will decide the amount of pharmaceutical composition or other active ingredient with which to treat each individual patient. Initially, the attending physician will administer low doses of pharmaceutical composition and observe the patient's response. Larger doses of pharmaceutical composition may be administered until the optimal prolphylactic effect is obtained for the patient, and at that point the dosage is not increased further. It is contemplated that the various pharmaceutical compositions used to practice the method of the present invention should contain about 0.01 gg to about 200 mg (preferably about 0.1 μg to about 100 mg, more preferably about 1 mg to about 20 mg, most preferably about 10 mg) of pharmaceutical composition or other active ingredient of the present invention per kg body weight.

Therapeutically useful agents other than a pharmaceutical composition or of the invention which may also optionally be included in the composition, may alternatively or additionally, be administered simultaneously or sequentially with the composition in the methods of the invention. Preferably for bone or cartilage protection potential materials are biodegradable and biologically well-defined, such as bone or dermal collagen.

### Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in models to achieve a circulating concentration range that includes the IC50 as determined in cell culture (i.e., the concentration of the test compound which achieves a half-maximal inhibition of the pharmaceutical composition's biological activity). Such information can be used to more accurately determine useful doses in humans.

A therapeutically effective dose refers to that amount of the compound that results in prevention of symptoms or disease in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 page 1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration. An exemplary dosage regimen for GAG-peptide complexes or compositions of the invention will be in the range of about 1 μg/kg to 200 mg/kg of body weight, with the preferred dose being about 1 mg/kg to 200 mg/kg of patient body weight, varying in adults and children. More preferably the dose is in the range 5-200 mg/kg, more preferably 10-100 mg/kg and more preferably 10-50 mg/kg, and most preferably 10-20 mg/kg. Dosing may be for example once daily, or equivalent doses maybe delivered at longer or shorter intervals. The amount or dose of composition administered will, of course, be dependent on the subject being treated, on the subject's age and weight, the manner of administration and the judgment of the prescribing physician.

### Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for prevention of an indicated condition.

It will be appreciated by persons skilled in the art that numerous variations and/or modifications may be made to the invention as shown in the specific embodiments without departing from the spirit or scope of the invention as broadly described. The present embodiments and examples are therefore to be considered, in all respects, as illustrative and not restrictive.

### EXAMPLES OF THE INVENTION

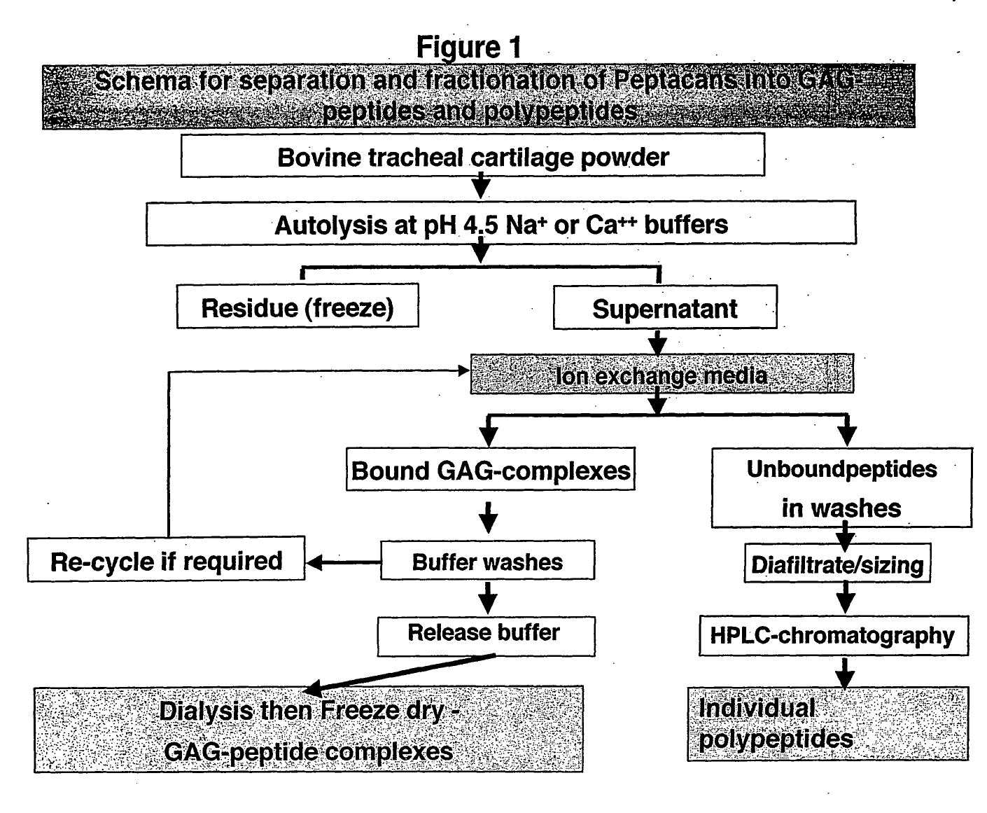

### Separation of Glycosaminoglycan Peptides (GAG-peptides) from Polypeptides in Peptacan Preparations by Ion-Exchange Solid Phase Media (see FIG. 1)

GAG-peptide and polypeptide mixtures released from cartilage using different buffers (eg. sodium or calcium acetate or dilute acetic acid) according to methods described in PCT/AU03/00061 are referred to as Peptacans. Calcium peptacan (CaP) is a GAG-peptide complex and polypeptide mixture released from cartilage by a method comprising autolysis using a calcium buffer. For separation, the freeze-dried Peptacans were dissolved in 0.1M calcium chloride buffered with Tris-HCl to a pH of 7.2 (application buffer) to afford sample concentrations of 4.0 mg/ml. To this solution was added pre-swollen DEAE-Sepharose-6B, or a similar medium, to achieve a final concentration of the ion exchanger of 100 mg/mL. The mixture was maintained at room temperature with gentle agitation for 16 hours in 5 mL stoppered centrifuge tubes. The tubes were then centrifuged at 1000 rpm for 5 mins and the supernatant decanted off. To the remaining pellet was added 1 mL of the application buffer and the tubes gently shaken, then centrifuged again as described previously. The supernatant washings were added to the original supernatant which were then subjected to ultra diafiltration using a 0.5 kDa cut-off membrane (YC05, cellulose acetate, Millipore Australia Pty Ltd. Sydney, Australia) in a stirred cell under nitrogen gas (50 psi) to remove the inorganic salts. The de-salted GAG-peptide complex solution was then freeze-dried and stored at −20° C. The GAG-peptide complexes obtained by this method were analysed for their protein and sulfated glycosaminoglycan (S-GAG) contents using the standard methods as described below. Using Calcium peptacan (CaP) as the starting material the pure GAG-peptide complex prepared by this ion-exchange procedure was identified by gel permeation chromatography and Composite Agarose Polyacrylamide Gel Electrophoresis (CAPAGE) to contain mainly 2 ChS chains attached to a short peptide stub and was annotated as GAG-P for all subsequent experiments.

### Preparation of a GAG-Peptide Complex by Limited Hydrolysis

Preparation of non-hydrolysed tracheal cartilage proteoglycans as a chromatography standard comprised mincing freshly cleaned bovine tracheal cartilage (3 mmn) and suspending 10 grams of the mince in 100 mLs of aqueous 4M guanidinium chloride (pH 5.8) at 4° C. for 48 hours as described by Inerot and Heinegard [Inerot S and Heinegard D, Bovine tracheal cartilage proteoglycans. Variations in structure and composition with age. Collagen and Related Research, 3: 245-262, 1983]. The guanidinium chloride and other inorganic salts were dialysed out, the water was removed by freeze-drying and the extracted proteoglycans were obtained as a white powder.

Hydrolysis using Bromelain comprised taking one kilogram of freshly cleaned and minced bovine tracheal cartilage, and subjecting the minced cartilage to 5 L of purified water containing 10 grams of sodium bicarbonate and 5 grams of Bromelain maintained at 60° C. and a pH of 4.8. After 24 hours the mixture was neutralised, filtered and freeze dried to yield 287 grams of a white powder. The composition and molecular weight distribution of the GAG-peptide complex in this preparation was determined by the assay methods described below.

Hydrolysis using aqueous sodium hydroxide comprised suspending aliquots of freeze-dried tracheal cartilage powder (10 grams) in 100 mLs of aqueous sodium hydroxide at concentrations varying from 0.1-2.0%. The stirred suspensions were incubated at 37° C. for 4, 8, 16, 24, 26, 28, 30 or 44 hours. Mixtures were adjusted to pH 7 with either acetic acid or ascorbic acid then filtered through a bed of diatomised earth (Celite). The resulting clear solutions were then subjected to Superdex-200 gel filtration chromatography to determine the size distribution and polydispersity of the GAG-peptide fractions released by hydrolysis.

### Fractional Separation of Glycosaminoglycan Peptides (GAG-Peptide Complexes) by Tangental Flow Ultrafiltration (TFF)

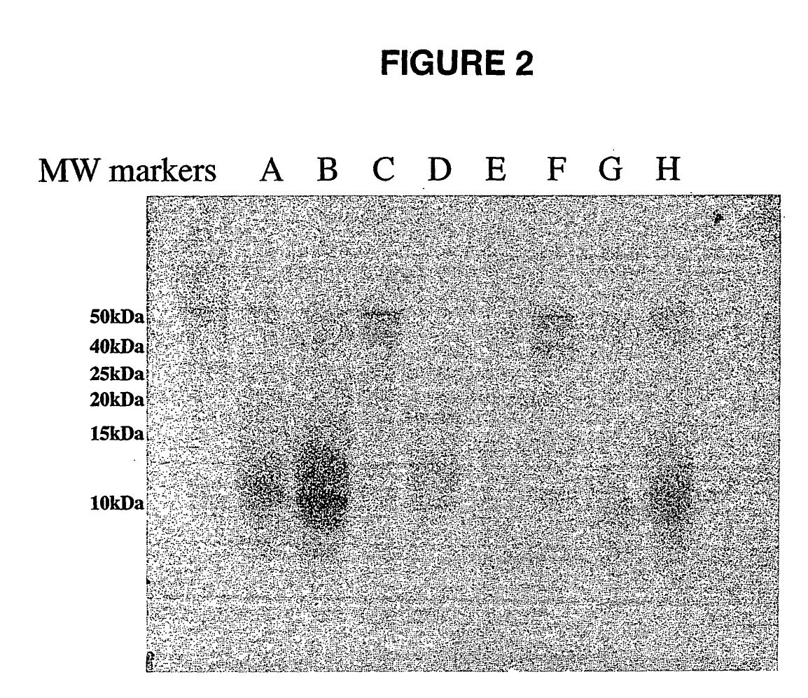

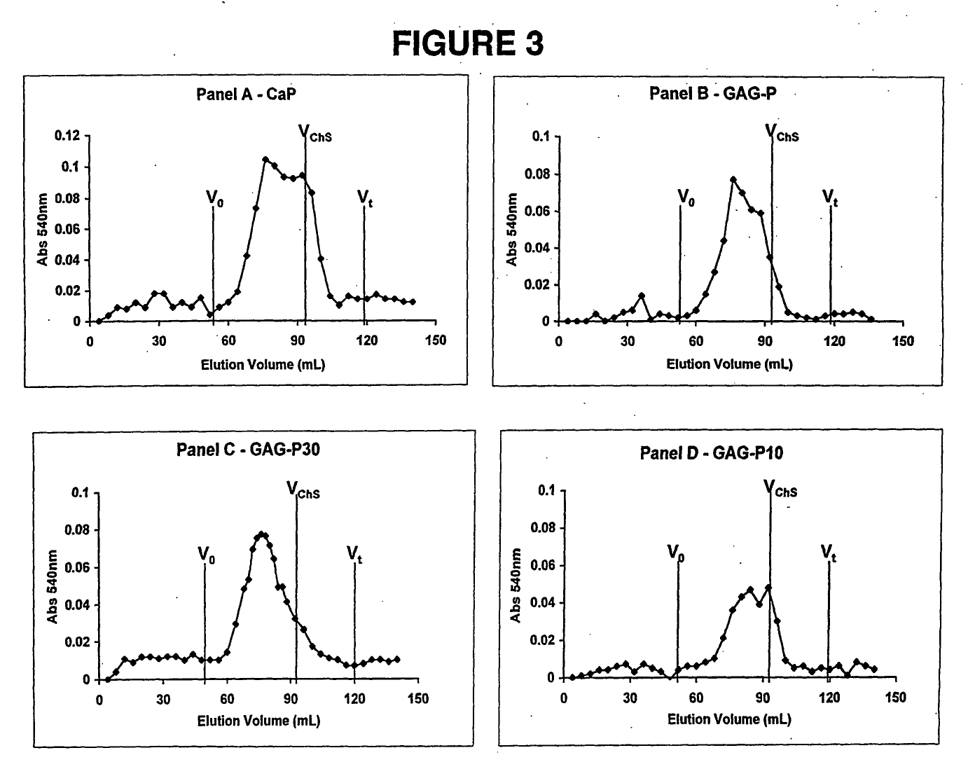

Subjecting aqueous solutions of mixtures of GAG-peptides and polypeptides prepared by the autolysis or limited hydrolysis to partial fractionation using tangential flow ultrafiltration (TFF) with membranes of different molecular weight cut-offs afforded GAG-peptide complexes within predetermined molecular size range. For example using a PLTC regenerated cellulose membrane with an exclusion size of 30,000 afforded a mixture of GAG-peptides and polypeptides in the retentate with molecular weights greater than 30,000 Da while the dialysate contained polypeptides with molecular weights less than 30,000 Da. The redistribution of polypeptides using this method was confirmed by SDS-PAGE (FIG. 2) while the size of the GAG-peptide complexes were determined by Superdex-200 chromatography (FIG. 3). The preparation isolated from the retentate was assigned the abbreviation GAG-P30 for subsequent experiments. Likewise, TFF fractionation of autolysis of limited hydrolysis solutions using a polyethersulfone spiral cartridge with an exclusion size of 10 kD afforded a mixture in the retentate containing GAG-peptides together with polypeptides with molecular weights >10,000 Da. This preparation was assigned the abbreviation GAG-P10 in subsequent experiments.

Calcium Peptacan was also sub-fractionated using ultrafiltration membranes of other molecular weights such as membranes with cut-offs of 20 or 1 kDa, the preparations so obtained being annotated as GAG-P20 and GAG-P1 respectively. These preparations were analysed for their protein and sulphated glycosaminoglycan (S-GAG) contents and molecular weight distribution using the standard methods described below.

### Analysis of Polypeptides Separated by Ion Exchange Using SDS-Polyacrylamide Gel Electrophoresis (SDS-PAGE)

Freeze dried polypeptide samples were dissolved in H₂O and then mixed 1:1 with 2× sample loading buffer (0.07 M TrisHCl, 1.5% SDS, 20% glycerol, 0.2M DTT and 0.1% BPB) to achieve the final concentrations of 4.0-20 mg/ml. The samples were boiled in a water bath for 5 min. 20 μl of above samples were loaded into the wells of 8-16% pre-cast Tris-glycine gel (Norvex). SeeBlue pre-stained low molecular weight range protein markers (Norvex) were loaded into wells on the left-hand side of the gel and electrophoresis was performed at 125 V for 2 h. The gel was stained in Coomassie blue R250 solution (40% ethanol, 10% acetic acid and 0.2% Coomassie R250) for 30 min and de-stained in a solution containing 10% ethanol and 7.5% acetic acid for 16 h. The gel was stored as a digitalised electronic image then dried in a Bio-Rad Gelair drier.

Analysis of Hydrolysed Tracheal Cartilage and GAG-Peptide Complexes from Membrane Diafiltration Using Superdex-200 Gel Permeation Chromatography

Aliquots (0.5-1 mL) of hydrolysis solutions or CaP preparations subjected to the TFF procedure were applied to a pre-packed 34×2 cm Superdex-200 (Pharmacia, Sydney, Australia) chromatography column in 0.25M NaCl. The column was eluted with 0.25M NaCl at a flow rate of 1.0 mL/minute. Fractions (1.0 mL) were collected and assayed for the levels of sulfated glycosaminoglycans using the method of Farndale et al as described below. The column void volume (Vo) and total volume (Vt) were determined using Dextran 2000 and radioactively labelled sulfate ion respectively. The elution volume for ChS was determined for a pharmaceutical grade preparation obtained from Bioiberica Ltd, Barcelona, Spain. The exclusion volume for purified non-hydrolysed tracheal cartilage PGs was also determined using the preparation described above.

### Sulfated Glycosaminoglycan (S-GAG) DMMB Assay

The total S-GAG content of samples was determined by binding to the metachromatic dye 1,9-dimethylmethylene blue (DMMB) [Farndale R W, Buttle D J and Barrett A J. Improved quantitation and discrimination of sulfated glycosaminoglycans by use of dimethylmethylene blue. Biochim. Biophys. Acta: 883, 173-177, 1986]. A standard curve was prepared using a commercially available chondroitin sulfate A (ChSA) derived from bovine tracheal cartilage (ICN USA) in 96-well microtitre plates. ChSA standard and Peptacan samples were diluted in 0.2% sodium formate before DMMB reagent was added and the absorbance at 535 nm read immediately. Softmax software was used to construct a standard curve and calculate the concentration of S-GAG in samples.

### Composite Agarose Polyacrylamide Gel Electrophoresis (CAPAGE)

Standard ChSA and GAG-peptide samples prepared by the methods described herein were dissolved in H₂O at the concentrations of 1.0-3.0 mg/ml and then mixed 1:1 with CAPAGE sample loading buffer (20 mM Tris-acetate, pH6.3, 1 mM Na2SO4, 60% sucrose and 0.01% bromophenol blue). Twenty micro litres of each sample, equivalent to 10 μg of GAG, was loaded into wells of 2 mm thick CAPAGE gel (0.6% agarose, 1.2% acrylamide, 10 mM Tris-acetate pH 6.3 and 0.25 mM sodium sulfate) and electrophoresed in the CAPAGE running buffer (10 mM Tris-acetate pH 6.3, 0.25 mM Na2SO4) at 150 V for 2 h. The gel was stained in a solution of 0.02% toluidine blue in 0.1 M acetic acid for 1 h, de-stained in 0.5 M acetic acid for 2 h and dried on an agarose gel-bound film. The dried gel was rinsed with H₂O, scanned and digitalised as an electronic image then dried in a Bio-Rad Gelair drier for storage.

Determination of the Concentration Dependent Anticoagulant Effects of CaP, GAG-Peptide Complex Preparations and Commercial Chondroitin Sulfate Preparations as Assessed from the Activated Partial Thromboplastin Time (APTT)

Pharmaceutical quality chondroitin sulfate were obtained from Bioiberica, Barcelona, Spain (ChS#1) and Sigma Chemical Co St Louis, Mo., USA (ChS#2). Calcium peptacan (CaP), and GAG-peptide (GAG-P), prepared as described herein, were evaluated for their anticoagulant activities using a commercial aPTT reagent (Actin FSL activated PTT reagent, Dade-Behring Margurg GmbH, Marburg, Germany) and a standard human plasma: (Ci-Trol Coagulation Control level 2, Dade-Behring Margurg GmbH, Marburg, Germany) according to the protocol supplied by the manufacturer. Stock solutions of the test preparations were dissolved in 0.025M calcium chloride and serially diluted in this buffer to correspond to the concentrations of 0-1.25 mg/mL. The aPTT times were determined by the addition of these solutions to a mixture of the Actin FSL reagent and control plasma held at 37° C. in cuvettes of a fibrin-timer (Dade-Behring Margurg GmbH, Marburg, Germany). This instrument quantitated the time in seconds for clot formation. All samples were assayed in duplicate and the mean values plotted against concentration using a semi-log scale.

### The Effects of CaP, GAG-Peptide Preparations, Glucosamine, Type II Collagen and Chondroitin Sulfate in Rat Models of Arthritis

A description of the method used to induce arthritis in the rat CIA and AIA models is provided below:

### The Rat Type II Collagen Induced Arthritis (CIA) Model

Female Wistar rats (160-180 gm) were inoculated with 250 μg of the arthritogen, bovine tracheal collagen type-II dissolved in dilute acetic acid (0.01M) and applied as 6 divided injections into their tail base on day 0. The body weight of animals, together with their tail and paw swelling, were determined every 48 h, the latter parameters being determined using callipers and results expressed in millimetres (mm). Arthritis development was also assessed from day 11 onwards using a macroscopic scoring scale whereby rear paw, fore paw, and tail swelling were scored on a scale 0-4+. An overall arthritis score (0-4+) was also determined on the basis of overall inflammation and other signs of disease activity, e.g. piloerection, diminished mobility, poor grooming etc as described previously [Creamer M. et al. Collagen-induced arthritis in rats. J Immunology, 149:1045-1053, 1992].

### Rat Adjuvant Arthritis (AIA) Model

Polyarthritis was induced in adult (180 g) Wistar rats on day 0 by the subdermal injection of the arthritogen, Mycobacterium tuberculosis (Mtb) (Difco Laboratories, Detroit, Mich., USA) (1 mg) in squalane (2,6,10,15,19,23-hexamethyltetracosane, Sigma, St Louis, USA) (0.1 mL) into the base of their tails on day 0. The body weight of animals, together with their tail and front and rear paw swelling, were measured every 48 h, using the same procedures as described for the CIA rat model. Arthritis development was determined from day 11 onwards using a macroscopic scoring of rear paw, fore paw and tail swelling, (on a scale 0-4+) and an overall arthritis score (also scored 0-4+) assigned on the basis of overall inflammation and other signs of disease activity, e.g piloerection, diminished mobility, poor grooming etc. as described in detail elsewhere [Whitehouse M W, Adjuvant induced polyarthritis in rats. In: Greenwald R A, Diamond H S, eds. Handbook of models for Rheumatic Diseases Vol 1. Boca Raton; CRC Press, 1988; 3-16].

### Treatment Protocol

Preparations were examined at various oral doses for their abilities to (i) tolerize animals against arthritis occurring by dosing the animal for 7 days prior to inducing arthritis by the injection of the arthritogen; (ii) preventing arthritic disease occurring by administering the compounds to the animals from the time they were injected with the artluitogen and for 15 days thereafter, i.e. a prophylactic protocol. These protocols are summarised in FIG. 4. All preparations were dissolved in de-ionised water and were administered to animals by gavage at doses between 3.3 mg/kg to 200 mg/kg body weight at the time intervals shown in FIG. 4.

### Topical Anti-Inflammatory Activity of GAG-Peptide Preparations in Human Subjects

The topical anti-inflammatory activity of the GAG-peptide preparations were evaluated in a standard chemically induced erythema test over an 8 day period. In the example presented here, a GAG-peptide preparation obtained by limited alkaline hydrolysis of bovine tracheal cartilage (GAG-PLH) was used. The GAG-PLH preparation was formulated as a 5% active in a standard cream base which contained glycerin, diisopropyl adipate, octyl salicylate, isopropyl adipate, isopropyl palmitate, stearic acid, cyclomethicone, xanthan, carbomer, allantolin, preservatives and water.

The same cream base without the GAG-PLH active was used as the placebo. The study was conducted under double blind conditions in which neither the test subject nor the assessor of the erythema score were aware of the identity of the cream applied. The subjects' left arm or right arm were randomly assigned to receive either the cream base (placebo) or the cream base plus active (active) so each subject provide his or her own control. The design of the study complied with the Helsinki criteria for experimental studies on humans and was approved by the Institutional Ethics Committee (IEC).

Before commencing the study test subjects were instructed to refrain from applying any personal care or therapeutic products to the arm test area for 7 days prior to starting the investigation. Eleven subjects qualified for inclusion in the study and were enrolled but one failed to complete the final visit and was therefore excluded from the study.

### Protocol

On the initial day of entry into the study the test sites on the right and left arms were wiped clean with water only. Park-David Readi Bandage occlusive patches (2 cm×2 cm) were impregnated with varying concentrations of sodium lauryl sulfate over the range of 0.25% to 2.0%. Similar test sites of both the inside forearms of each subject were selected and covered with a series of the prepared patches so as to elicit a graded chemically induced erythemal response. All patches were removed 22 to 26 hours after application. One hour after removal of the patches all sites were scored, using the scoring was assigned numerical values as shown below:
0=no evidence of any effect (Value=0)
?=query (Value=1)+
+1=minimal, faint, uniform or spotty erythema (Value=2)
1=pink uniform erythema covering most or all of the contact site (Value=3)
2=pin-red erythema visibly uniform in entire contact site (Value=4)
3=bright red erythema with or without petechiae or papules (Value=5)
4=deep red erythema with or without vesiculation or weeping (Value=6)

Subjects were then instructed to apply either the coded placebo or the active (as defined above) undiluted creams, twice daily, morning and evening for eight days to either the erythema sites on the right or left arms. The response to treatments was assessed using the same grading system as undertaken initially as described above. Evaluation of response was then repeated at each subsequent time point i.e. 2, 4, 8 days after the initial application of the creams.

After breaking of the blinded code the individual mean responses for the 4 times points for each of the preparations was determined and compared. The Student's paired t-Test and null hypothesis were then used to evaluate the data and determine whether differences existed between the two treatment groups. P<0.05 was considered to be statistically significant.

### Results and Discussion

Using a GAG-peptide complex polypeptide mixture obtained by autolysis (CaP) as an example and the protocol shown schematically in FIG. 1, a calcium salt of a glycosaminoglycan peptide (GAG-P) consisting largely of 2 ChS chains covalently attached to a peptide stub and free of protein or peptides was obtained as confirmed by chemical analysis, PAGE and gel filtration chromatography. The TFF ultrafiltration method, using various membranes with 0.5 kDa, 1.0 kDa, 10 kDa and 30 kDa molecular weight cut off, also provided a means of partially purifying the products of cartilage autolysis and limited hydrolysis. However, the technique not only selectively removed proteins and peptides from the preparations, as shown in FIG. 2, but also fractionated the GAG-peptides present according to the molecular size distribution (see FIG. 3). When the GAG-peptide complex polypeptide mixture was prepared from tracheal cartilage using acetic acid buffer containing no monovalent or divalent cations (see patent PCT/AU03/00061) was subjected to the same ion exchange procedure to that shown in FIG. 1, a GAG-peptide complex containing 3 ChS chains was obtained.

It was also demonstrated that GAG-peptide complexes of the desired molecular size could be obtained from cartilage using the procedure of limited hydrolysis of cartilage. This was illustrated using the proteolytic enzyme, bromelain or by alkaline hydrolysis with sodium hydroxide as examples.

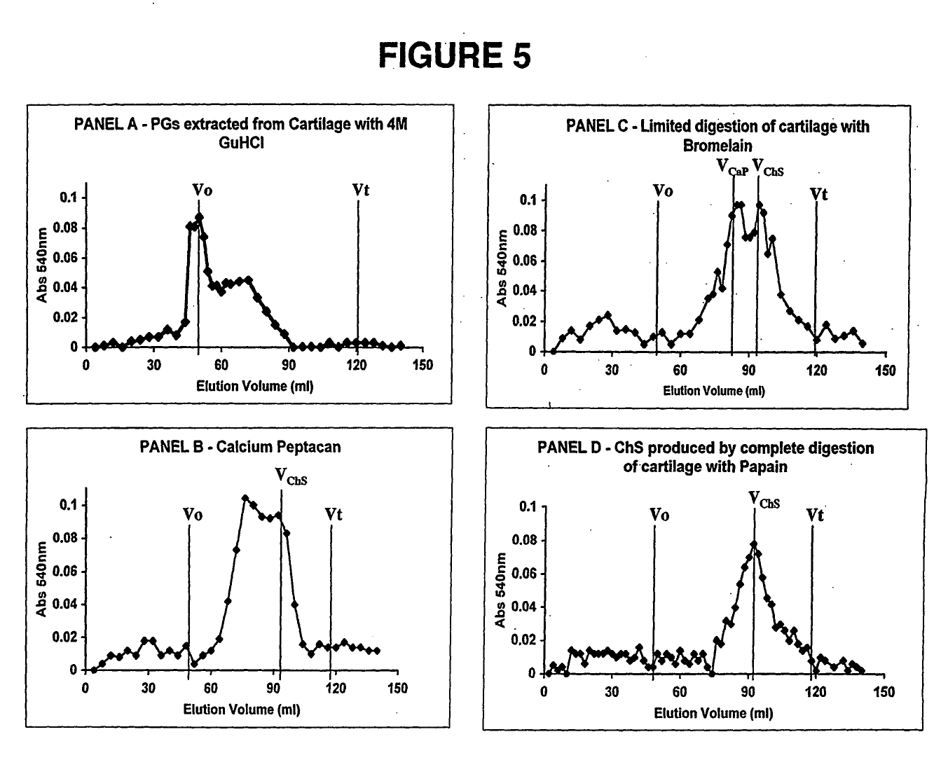

As shown in FIG. 5, under the conditions described herein the proteoglycans present in bovine tracheal cartilage (FIG. 5, panel A) are only partially degraded to ChS by bromelain. The other polyanionic species produced corresponding to GAG-peptide complexes of similar molecular size to those of CaP (compare FIG. 5, panels B and C).

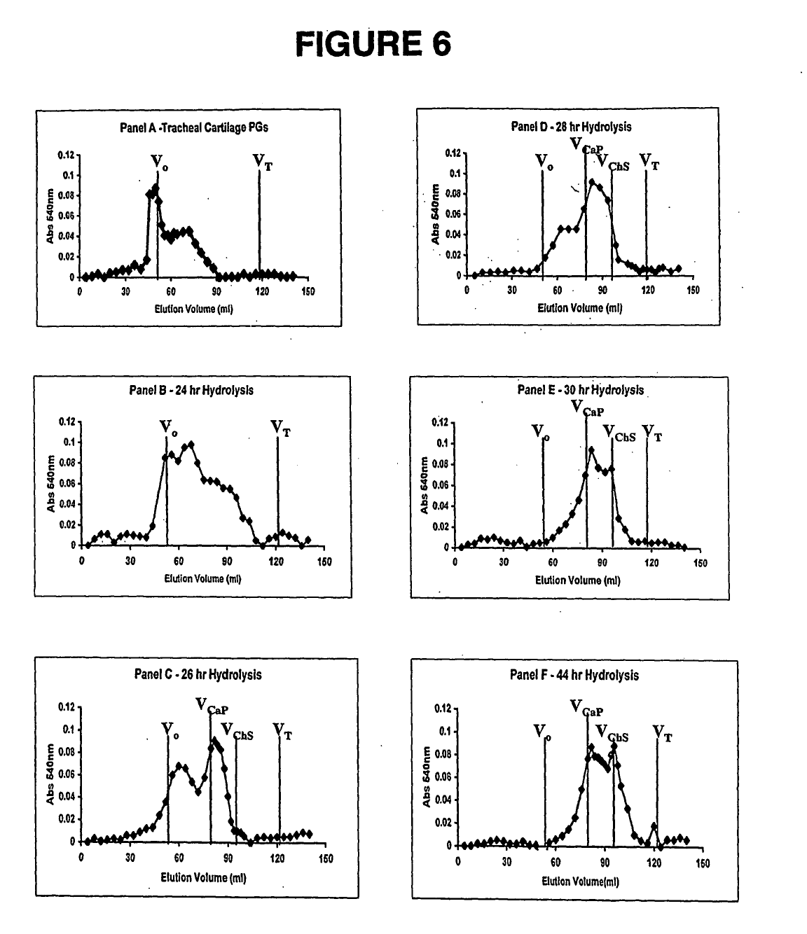

Limited hydrolysis of bovine cartilage with solutions of aqueous sodium hydroxide were found to undergo a more complex pathway of breakdown with molecular species of similar size to CaP being generated between 26 and 44 hours at 37° C. However, the longer hydrolysis times were found to increase the proportions of ChS present (FIG. 6, panels C-F).

Even though these experiments have shown that bromelain or sodium hydroxide can be used to produce a GAG-peptide complex from cartilage with a molecular size similar to or greater than CaP, these preparations would appear to also contain an amount of single chain ChS. As shown in FIG. 3 the smaller GAG species, including ChS can be removed from these mixtures by TFF ultra filtration with appropriate membranes.

Pharmacological Activities of GAG-Peptide Complexes

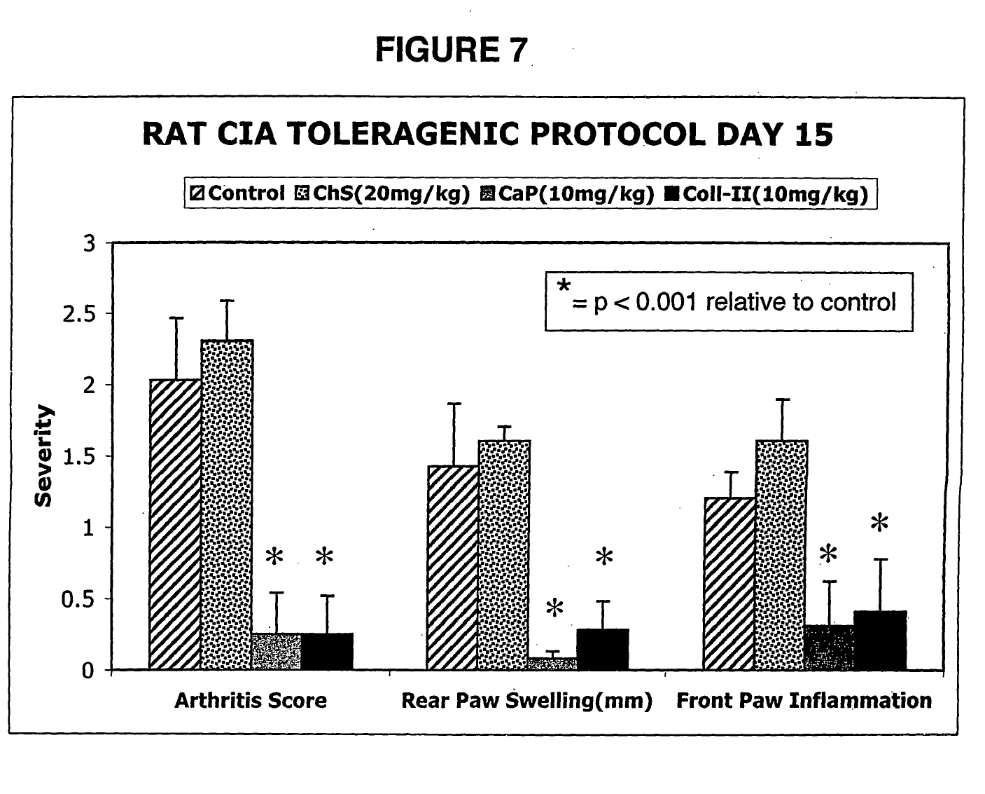

One of the most unexpected finding arising from the present investigations was the observation that oral administration of CaP or the GAG-P complex alone prepared as described herein to rats for 7 days before inducing CIA suppressed the manifestations of the disease for up to 18 days post antigen inoculation. The tolerization of these animals against disease development by CaP at 10 mg/kg was equivalent to the tolerizing effects of type II collagen when administered at the same dose (FIG. 7). Type II collagen is a well known toleragen for CIA but is also an arthritogen when injected back into the animals with adjuvant [Creamer M, et al, Collagen-induced arthritis in rats, J Immunology, 149:1045-1053, 1992, and other references cited herein].

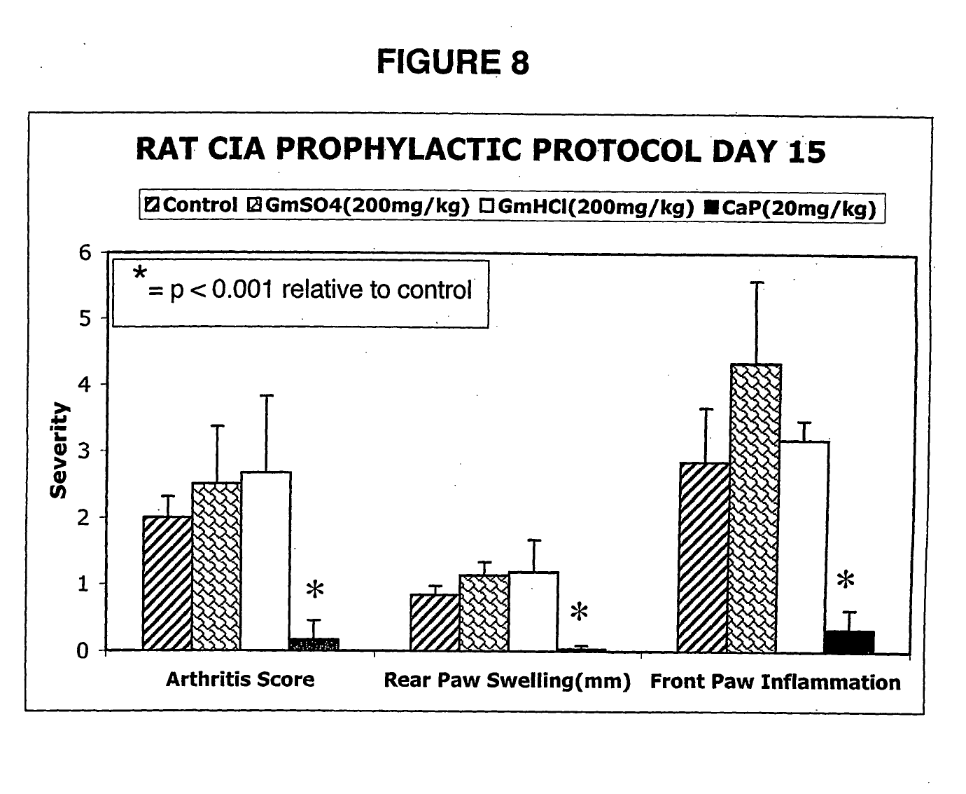

In contrast to type II collagen, CaP was not arthritogenic when used to re-challenge the pre-treated by injection into the base of their tails with adjuvant (results not shown). The results shown in FIG. 7 also confirmed the earlier findings of Omata et al (2000), [Effects of chondroitin sulfate-C on articular cartilage destruction in murine collagen-induced arthritis. Arzneim Forsch./Drug Res. 50: 148-153:2000] that ChS was inactive as a toleragen in the CIA model. Moreover, a comparative study of CaP with other neutraceuticals currently used to treat OA, glucosamine sulfate and glucosamine hydrochloride showed that the latter were inactive as toleragens in the rat CIA model [FIG. 8].

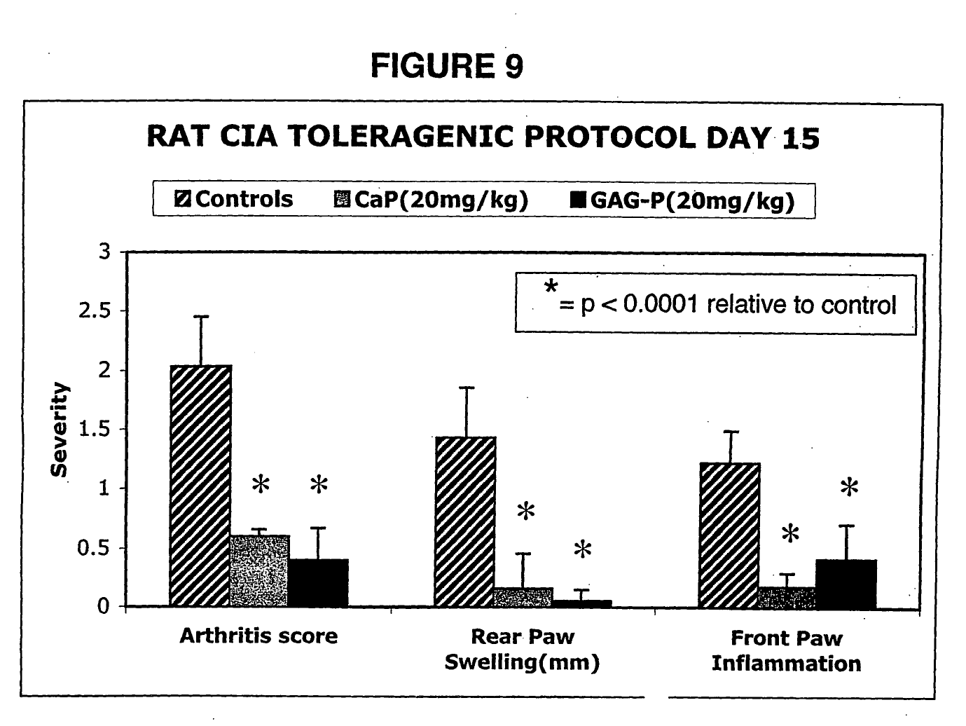

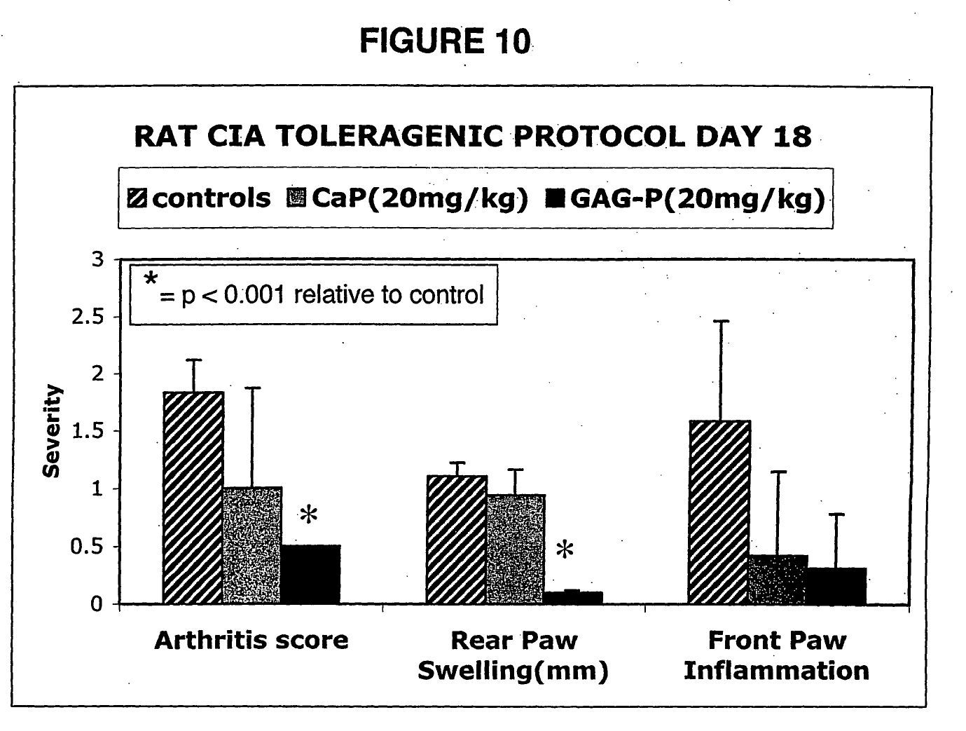

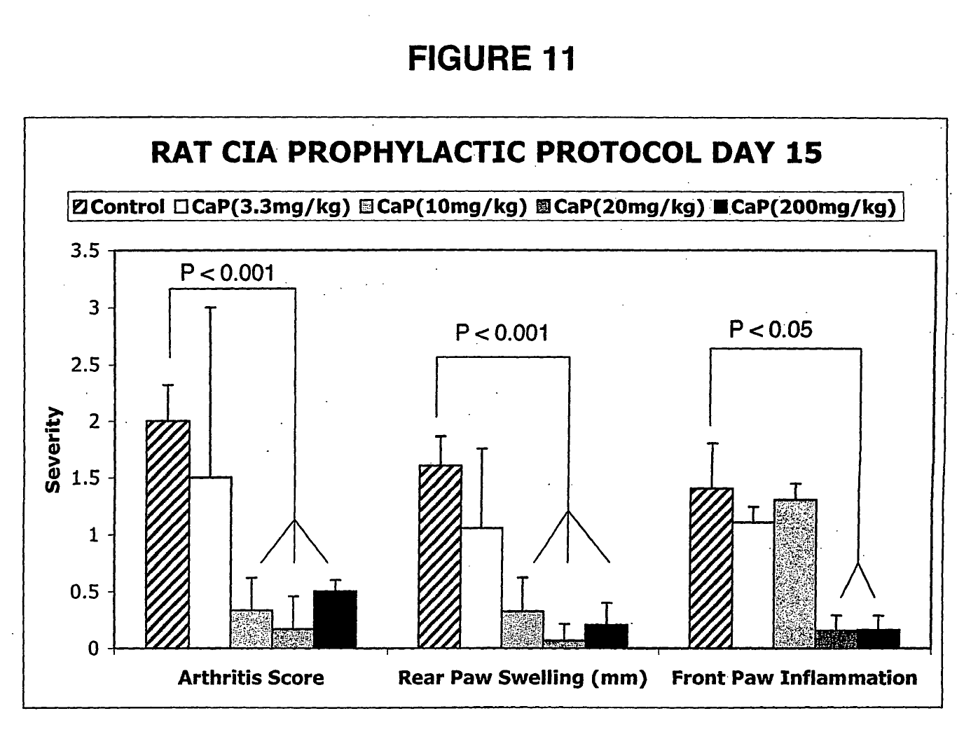

The GAG-peptide complex (alone) prepared from CaP by the ion exchange method [FIG. 1] was demonstrated to be a potent toleragen in the CIA model (FIG. 9) and seemed to have a longer lasting effect than CaP when used at the same dose of 20 mg/kg as indicated from the results obtained on day 18 (FIG. 10]. In addition, a dose ranging study using the CIA model demonstrated that CaP was active as a toleragen at and 200 mg/kg but not at 3.3 mg/kg [FIG. 11].

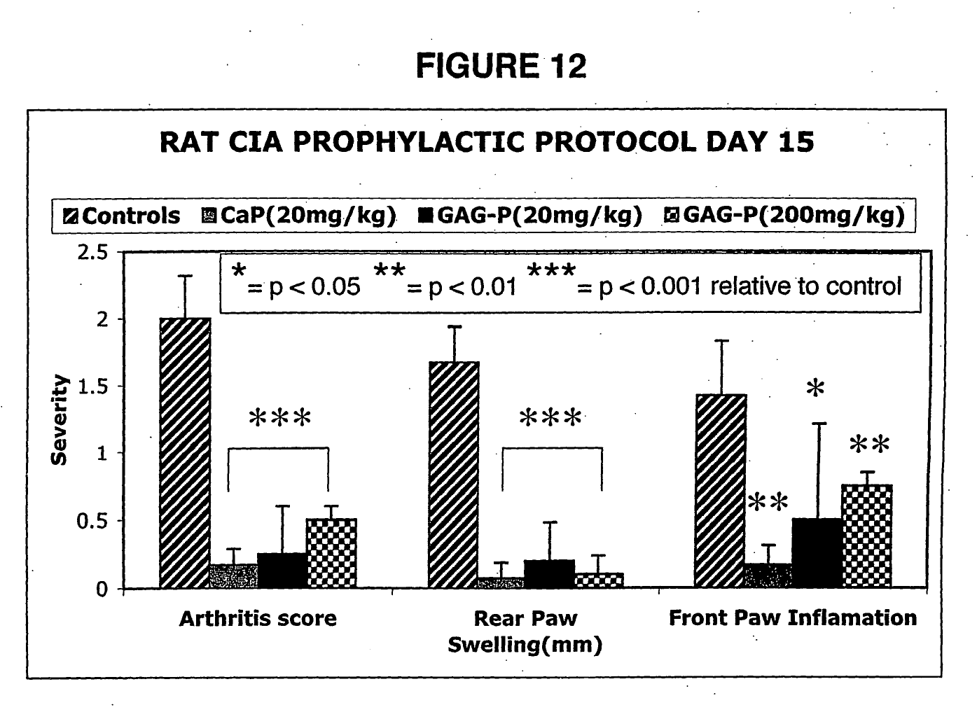

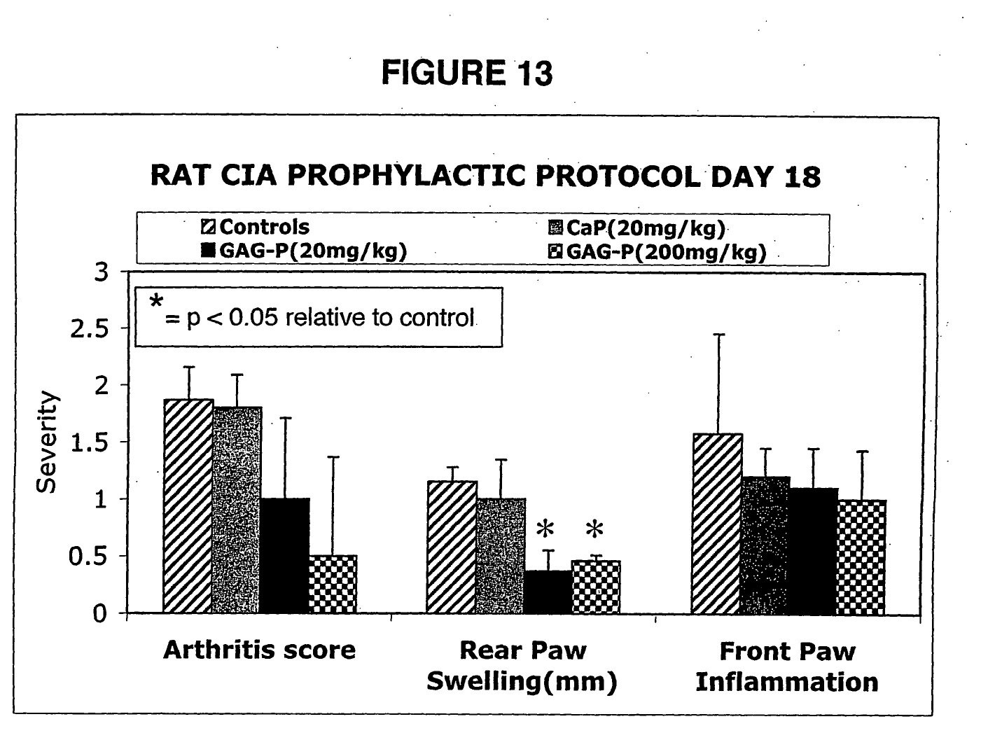

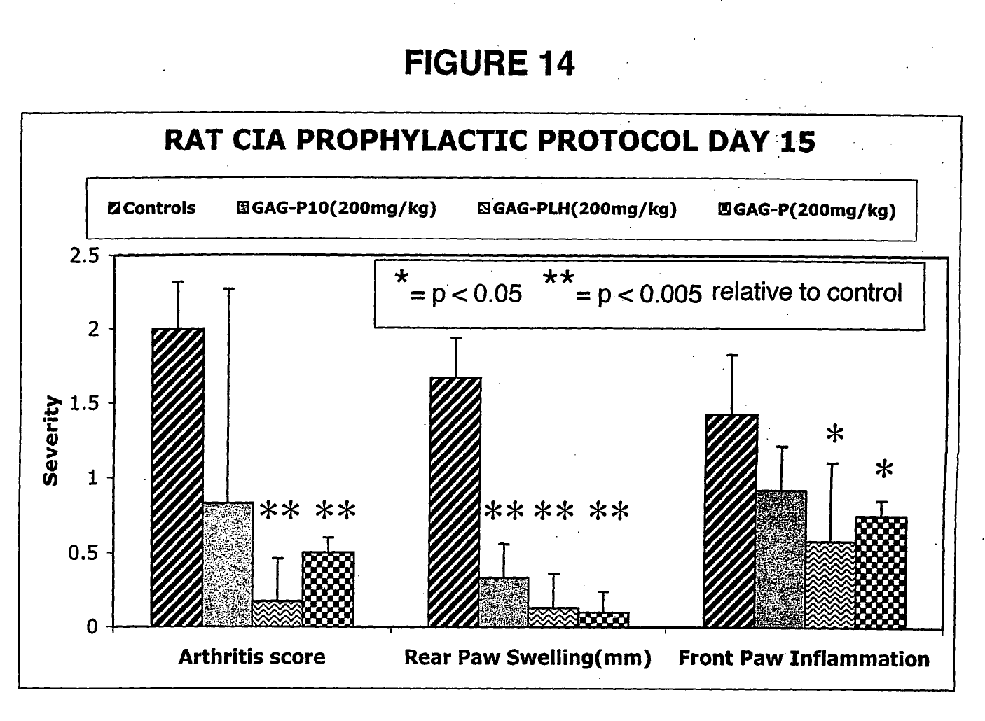

CaP and GAG-P were also shown to be equi-potent in the rat CIA model when administered using the prophylactic protocol [FIG. 12]. However, by day 18 CaP at 20 mg/kg was found to be less effective than GAG-P as suggested by the extent of rear paw swelling [FIG. 13]. While the GAG-peptide prepared from cartilage by limited hydrolysis with sodium hydroxide (GAG-PLH) was found to be equivalent to GAG-P in the rat CIA model when given prophylactically at 200 mg/kg, the smaller GAG fraction, GAG-P10, was less active at the same dose [FIG. 14].

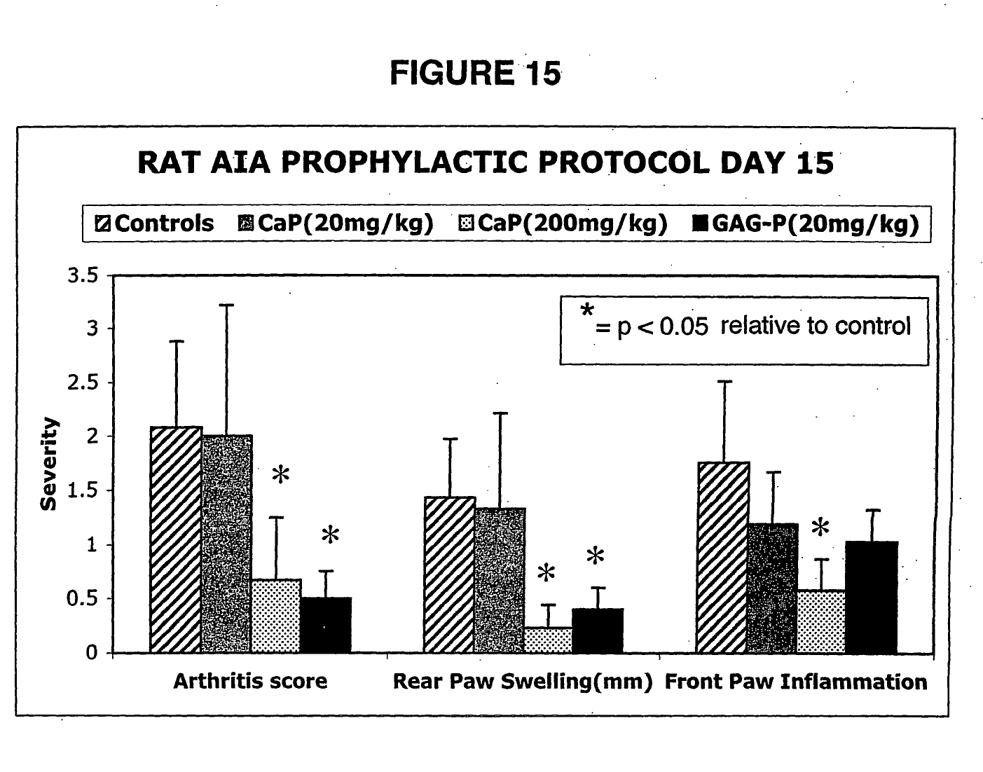

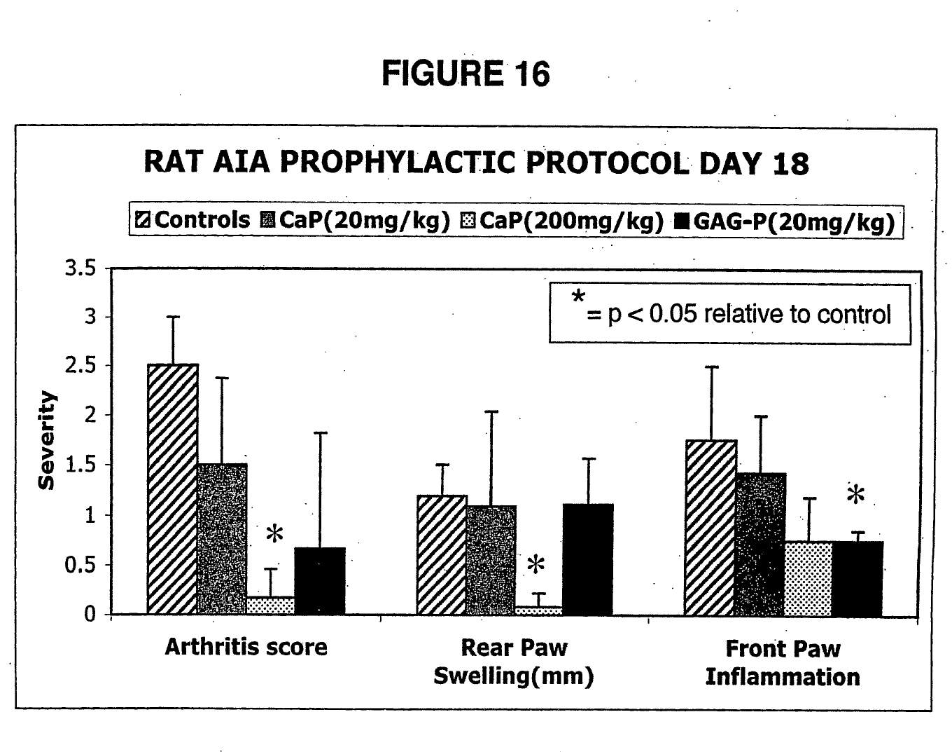

Both CaP and GAG-P were active in preventing disease development when used prophylactically in the rat AIA model [FIG. 15]. However, a stronger effect of GAG-P was indicated on Day 18 in terms of front paw inflammation, although CaP at 200 mg/kg demonstrated higher potency in the other parameters [FIG. 16].

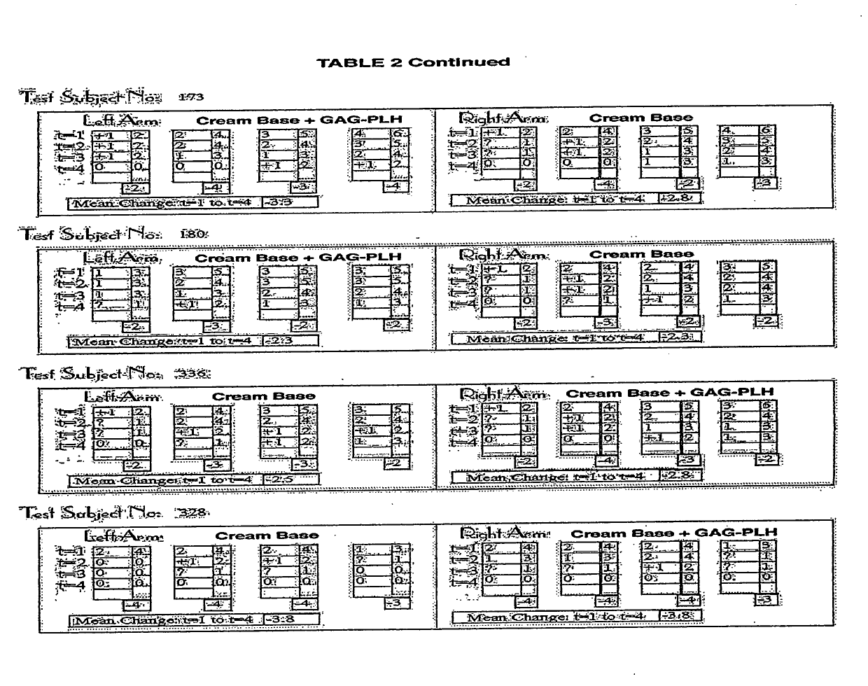

As discussed earlier the chronic use of NSAIDs by OA and RA patients is frequently associated with serious side effects in the gastrointestinal tract particularly with respect the induction of gastric bleeding. This effect was confirmed in an animal model by administering 50 mg/kg of the NSAID, ibuprofen, to rats in which CIA had been ongoing for 20 days [see FIG. 18—Table 1]. The gastro protective ability of CaP was demonstrated in this model when it was administered prophylactically at 20 or 200 mg/kg for 15 days, or even for 4 days before giving the NSAID [Table 1]. In this animal model the commonly used anti-arthritis drug, aurothiomalate, was only marginally effective [Table 1]. The reduced ability of CaP and GAG-P to promote bleeding was also demonstrated using the anti-coagulant assay [FIG. 17]. In this assay ChS exhibited anti-coagulant activity over the concentration range of 0.4-1.2 mg/mL while CaP and GAG-P demonstrated marginal effect at the same concentrations [FIG. 17].

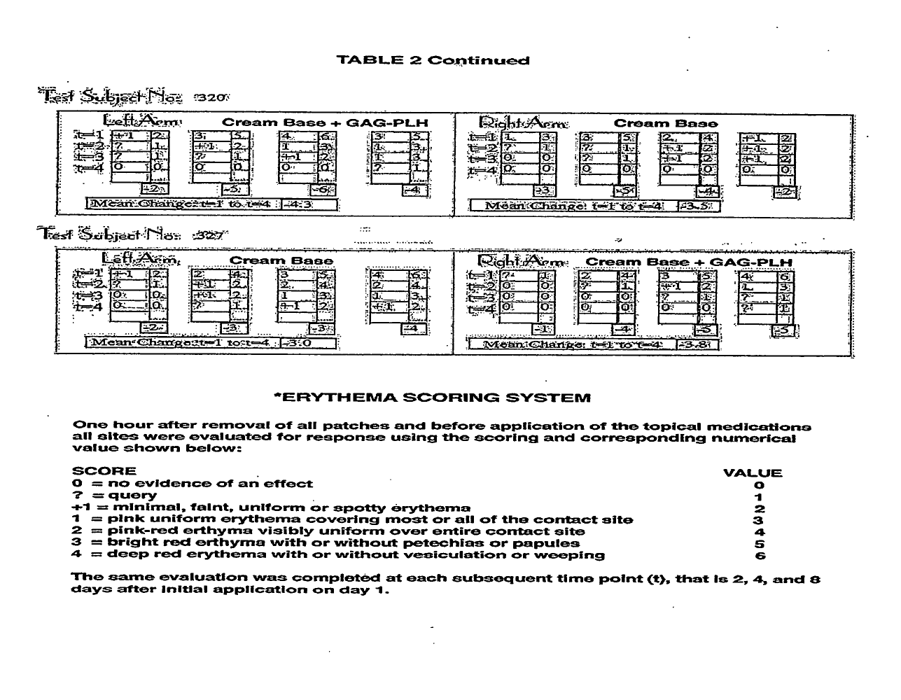

The GAG-peptides described in this application also demonstrated anti-inflammatory activity when applied topically in a cream base to human subjects. The results of a study undertaken with GAG-PLH are shown in Table 2 [FIG. 19]. The results of this study showed that 8 out of the 10 subjects who used the cream base with GAG-PLH experienced a positive response, the remaining 2 subjects exhibiting equivalent activity to the cream base alone [Table 2]. The mean value and standard deviation for the 10 subjects who applied the cream base containing GAG-PLH to the erythema site was determined to be 3.04±0.82 while the corresponding values for the subjects applying cream base alone was, 2.59±0.88. Analysis of these data using the paired values for each subject (left or right arms) showed that the two cream treatments were statistically different at the p<0.002 probability level.

The above study serves to demonstrate that the GAG-peptide complexes described herein were not only active in preventing inflammation and arthritis in animal models of arthritis when administered orally but were also active when applied topically in human subjects.

1. A method of preventing the onset of a harmful immune response or at least one symptom thereof in a connective tissue of an animal to an antigen, comprising administering the animal with an effective amount of a composition comprising one or more GAG-peptide complex, wherein at least one GAG-peptide complex comprises 2 or 3 GAG chains.

2. The method according to claim 1, wherein the at least one symptom is selected from the group consisting of inflammation, cell injury, tissue injury, tissue degradation, redness, tenderness, swelling, joint stiffness, impaired mobility, impaired strength or combinations thereof.

3. The method according to claim 1 wherein the connective tissue is a connective tissue of skin, tendon, ligament, cartilage, bone, fat tissue or combinations thereof.

4. The method according to claim 1 wherein the method comprises a prophylactic protocol.

5. The method according to claim 1, wherein the harmful immune response is prevented.

6. A method of prophylaxis to prevent a harmful immune response in an animal to an antigen or at least one symptom thereof in a connective tissue of the animal, comprising administering the animal with an effective amount of a composition comprising one or more GAG-peptide complexes wherein at least one GAG-peptide complex comprises 2 or 3 GAG chains.

7. The method according to claim 1, wherein the method induces tolerance in the animal to the antigen.

8. A method of inducing tolerance to prevent a harmful immune response in an animal to at least one antigenic component of cartilage comprising administering the animal with an effective amount of a composition comprising one or more GAG-peptide complexes wherein at least one GAG-peptide complex comprises 2 or 3 GAG chains.

9. The method according to claim 1, wherein the immune response is an autoimmune response.

10. The method according to claim 1, wherein the composition further comprises at least one connective tissue derived polypeptide.

11. The method according to claim 1, wherein the composition is administered orally or topically.

12. The method according to claim 1, wherein the composition is administered orally.

13. The method according to claim 1 wherein the composition is administered topically,

14. The method according to claim 1, wherein the composition is administered in an amount of about 10-20 mg/kg.

15. Use of a composition comprising one or more GAG-peptide complex in the manufacture of a medicament for preventing a harmful immune response or the onset of at least one symptom thereof in a connective tissue of an animal to an antigen, wherein at least one GAG-peptide complex comprises 2 or 3 GAG chains.

16. Use according to claim 15, wherein the connective tissue is a connective tissue of skin, tendon, ligament, cartilage, bone, fat tissue or combination thereof.

17. Use according to claim 15, wherein at least one symptom is selected from the group consisting of inflammation, cell injury, tissue injury, tissue degradation, redness, tenderness, swelling, joint stiffness, impaired mobility, impaired strength or a combination thereof.

18. Use according to claim 15 wherein the method comprises a prophylactic protocol.

19. Use according to claim 15, wherein the harmful immune response is prevented.

20. Use of a composition comprising one or more GAG-peptide complexes in the manufacture of a medicament for prophylactic therapy of an animal to prevent a harmful immune response to an antigen or at least one symptom thereof in a connective tissue, wherein at least one GAG-peptide complex comprises 2 or 3 GAG chains.

21. Use according to claim 15, wherein the method induces tolerance in the animal to the antigen.

22. Use of a composition comprising one or more GAG-peptide complexes in the manufacture of a medicament for inducing tolerance in an animal to at least one antigenic component of cartilage to prevent a harmful immune response, wherein at least one GAG-peptide comprises 2 or 3 GAG chains.

23. Use according to claim 15, wherein the immune response is an autoimmune response to at least one antigenic component of cartilage.

24. Use according to claim 15, wherein the composition further comprises at least one connective tissue derived polypeptide.

25. Use according to claim 15, wherein the composition is formulated for oral or topical administration.

26. Use according to claim 15, wherein the composition is formulated for oral administration.

27. Use according to claim 15 wherein the composition is formulated for topical administration.

28. Use of a composition comprising one or more GAG-peptide complexes in the manufacture of a medicament for preventing an autoimmune response to at least one antigenic component of cartilage, wherein at least one GAG-peptide complex comprises 2 or 3 GAG chains.

29. A pharmaceutical composition suitable for preventing the onset of a harmful immune response or at least one symptom thereof in a connective tissue of an animal to an antigen said composition comprising one or more GAG-peptide complex in combination with a pharmaceutically acceptable carrier, wherein at least one GAG-peptide complex comprises 2 or 3 GAG chains.

30. The pharmaceutical composition according to claim 29 formulated for prophylactic therapy of an animal.

31. The pharmaceutical composition according to claim 29 formulated for inducing tolerance.

32. The pharmaceutical composition according to claim 28 comprising at least one connective tissue derived polypeptide.

33. The pharmaceutical composition according to claim 29 wherein the at least one GAG peptide has a molecular weight of greater than about 30,000 Da.

34. The pharmaceutical composition according to claim 29, wherein the GAG-peptide complex and connective tissue derived polypeptide are obtainable from a connective tissue by a method comprising.
(i) incubating a connective tissue in an autolysis medium that provides a buffered pH range of between about pH 2.5 and about pH 8.5 for a time and under conditions sufficient to release at least one GAG-peptide complex and at least one polypeptide; and
(ii) recovering a mixture comprising at least one GAG-peptide complex and at least one polypeptide from the autolysis medium.

35. The pharmaceutical composition according to claim 34, wherein the GAG-peptide complex and connective tissue derived polypeptide are obtainable from a connective tissue by a method comprising
(i) incubating a connective tissue in an aqueous alkaline hydrolysis medium for a time and under conditions sufficient to release at least one GAG-peptide complex and at least one polypeptide; and
(ii) recovering a mixture comprising at least one GAG-peptide complex and at least one polypeptide from the hydrolysis medium.

36. The pharmaceutical composition according to claim 34 wherein the GAG-peptide complexes are separated from the polypeptides and recovered.
